(19) United States
(12) Patent Application Publication
(10) Pub. No.: US 2026/0234362 A1
(43) Pub. Date: Aug. 13, 2026

(54) METHOD OF CONVERTING POLYMER COMPRISING BENZENOID RINGS INTO OXIDATION PRODUCTS

(71) Applicant: AGENCY FOR SCIENCE, TECHNOLOGY AND RESEARCH, Singapore (SG)

(72) Inventors: Yuan Chong Jason Lim, Singapore (SG); Albert Ong, Singapore (SG)

(21) Appl. No.: 19/156,037

(22) PCT Filed: Feb. 8, 2024
(86) PCT No.: PCT/SG2024/050068

§ 371 Date: Aug. 13, 2025

(30) Foreign Application Priority Data
Feb. 13, 2023 (SG) 10202300351V

(51) Int. Cl. C08J 11/28; C07C 51/16; C08J 11/16

(52) U.S. Cl. CPC C08J 11/28; C07C 51/16; C08J 11/16; C08J 2325/06

(57) ABSTRACT

There is provided a method of converting a polymer comprising benzenoid rings into oxidation product(s), the method comprising: a step of oxidizing a benzenoid-based polymer source by adding a NOX source to the benzenoid-based polymer source to oxidize the polymer comprising benzenoid rings. Preferably, the NOX source is selected from nitrous acid, nitrite salts, nitric acid, nitrate salts and combinations thereof. Preferably, the step of oxidizing is carried out in the presence of a catalyst comprising N-hydroxy (—N—OH) groups.

### TECHNICAL FIELD

The present disclosure relates broadly to a method of converting polymer comprising benzenoid rings into oxidation products.

### BACKGROUND

Polymers comprising benzenoid rings are one of the most common materials used in our daily life, with millions of tonnes produced each year. One such example is polystyrene (PS) which has a production scale of 16 million metric tonnes per year, accounting for about 6% of the global plastic market share. These polymers are used in a wide range of applications ranging from building materials, protective packaging, and electronics to daily used food containers, lids, bottles, trays such as Styrofoam.

Despite its wide range of applications, a significant fraction of polymers comprising benzenoid rings is intended for single-use applications, which resulted in a large amount of waste production. The accumulation of waste has been accelerating in recent years due to the global shift to single-use food and beverage containers and rise in the demand/trend for food delivery and takeaways especially since the Covid-19 pandemic.

Furthermore, polymers comprising benzenoid rings are often made up of saturated hydrocarbons (C—C bonds or backbones) with high molecular weights, making the benzenoid polymers chemically inert and hydrophobic, and therefore resistant to natural degradation (or biodegradation). Accordingly, due to their non-biodegradable nature, these polymers could remain and accumulate in the environment for hundreds or thousands of years, which would be detrimental to earth.

With increasing public awareness of pollution, efforts have been made to focus on recycling and/or upcycling, but it remains very challenging from both technological and economic standpoints. As a result, the recycling rate of benzenoid polymers (e.g., PS) is currently less than 1% and accounts for approximately one-third of the landfills in the world.

Current recycling and/or upcycling methods have several drawbacks and are far from desirable. In particular, current techniques revolve around the use of either thermal reactions such as pyrolysis or photodegradation, which have their respective disadvantages. Firstly, pyrolysis typically requires an inert atmosphere and high energy consumption/demand (e.g., high temperature >300° C. and pressure), leading to high costs. Next, although catalytic photoreactions (e.g., photodegradation) has been shown to give efficient yields, the reaction conditions and pathways involved in photoreactions tend to be either complex with a long degradation time or require the use of toxic solvents such as aromatic solvents (e.g., benzene) or chlorinated solvents (e.g., 1,2-dichloroethane or dichloromethane). Light-based photoreactions are also difficult to upscale due to the existence of common additives such as dyes or plasticizers in the polymers which can inhibit light penetration, thereby hindering photodegradation reactions, limiting the efficacy and types of polymer waste products which could be degraded/upcycled. A number of current methods are also dependent on the use of metal-containing catalysts which can contribute to heavy-metal pollution and lead to environmental pollution if irresponsibly disposed.

In view of the above, there is a need to address or at least ameliorate the above-mentioned problems. In particular, there is a need to provide a simple and environmentally sustainable method for recycling polymers comprising benzenoid rings waste.

### SUMMARY

In one aspect, there is provided a method of converting a polymer comprising benzenoid rings into oxidation product(s), the method comprising:
a step of oxidizing a benzenoid-based polymer source by adding a NOₓ source to the benzenoid-based polymer source to oxidize the polymer comprising benzenoid rings.

In one embodiment, the NOₓ source is selected from the group consisting of nitrous acid, nitrite salts, nitric acid, nitrate salts and combinations thereof.

In one embodiment, the NOₓ source is selected from the group consisting of nitric acid (HNO₃), sodium nitrate (NaNO₃), potassium nitrate (KNO₃), magnesium nitrate (Mg(NO₃)₂), calcium nitrate (Ca(NO₃)₂), barium nitrate (Ba(NO₃)₂), aluminium nitrate Al(NO₃)₃, copper(II) nitrate (Cu(NO₃)₂), zinc nitrate (Zn(NO₃)₂), tetra-n-butylammonium nitrate, cerium(III) nitrate (Ce(NO₃)₃), cerium(IV) ammonium nitrate (CAN) (NH₄)₂[Ce(NO₃)₆]), iron(III) nitrate (Fe(NO₃)₃), lanthanum nitrate (La(NO₃)₃) and combinations thereof.

In one embodiment, the step of oxidizing is carried out in the presence of:
(i) an oxygen-containing gas;
(ii) optionally an environmentally benign solvent; and
(iii) optionally a catalyst.

In one embodiment, the polymer comprising benzenoid rings comprises:
(R1) one or more benzenoid repeating units represented by general formula (1A); and
(R2) optionally one or more non-benzenoid repeating units:

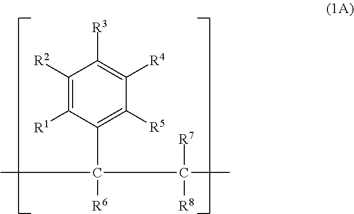

wherein
R¹ to R⁵ are each independently selected from the group consisting of H, optionally substituted alkyl, optionally substituted alkenyl, optionally substituted alkynyl, SO₃H, halogen atoms and combinations thereof;
R⁶ is H; and
R⁷ to R⁸ are each independently selected from the group consisting of H, optionally substituted alkyl, optionally substituted alkenyl, optionally substituted alkynyl and combinations thereof.

In one embodiment, the oxidation product(s) comprises organic acid functional group.

In one embodiment, the oxidation product(s) comprises one or more compounds represented by general formula (2):

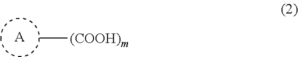

wherein
A is selected from the group consisting of H, optionally substituted alkyl, optionally substituted alkenyl, optionally substituted alkynyl, optionally substituted 5-membered ring and optionally substituted 6-membered ring; and
m=1 to 6.

In one embodiment, A is optionally substituted 6-membered ring and the oxidation product(s) comprises one or more compounds having general formula (2B):

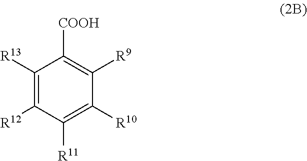

wherein
R⁹ to R¹³ are each independently selected from the group consisting of hydrogen (—H), nitro (—NO₂), carboxylic acid (—COOH), cyano (—CN), ester (—COOR), halogen (e.g., —F, —Cl, —Br, —I), hydroxy (—OH), alkoxy (—OR), sulfonic acid (—SO₃H), optionally substituted alkyl, optionally substituted alkenyl, and optionally substituted alkynyl, where R is an organic group.

In one embodiment, the oxidation product(s) comprises one or more of the following: terephthalic acid, phthalic acid, isophthalic acid, benzoic acid, nitrobenzoic acid and formic acid.

In one embodiment, the catalyst is present and comprises N-hydroxyl (—N—OH) groups.

In one embodiment, the catalyst comprises a structure selected from general formula (3), (4) and/or (5):

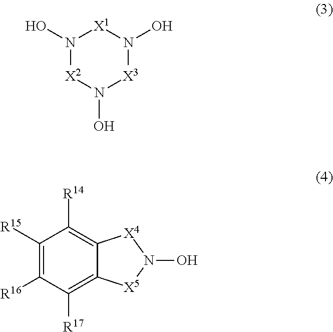

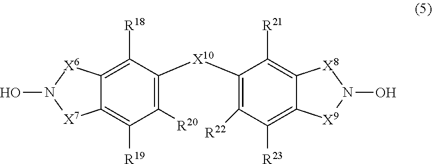

wherein
X¹ to X¹⁰ are each independently selected from the group consisting of CH₂, C(═O), S(═O)₂ and combinations thereof; and
R¹⁴ to R²³ are each independently selected from the group consisting of hydrogen (—H), nitro (—NO₂), carboxylic acid (—COOH), cyano (—CN), ester (—COOR), amine (—NR₂), halogen (e.g., —F, —Cl, —Br, —I), hydroxy (—OH), alkoxy (—OR), optionally substituted alkyl, optionally substituted alkenyl, optionally substituted alkynyl, and combinations thereof, where R is an organic group.

In one embodiment, the environmentally benign solvent is present and comprises a weak acid.

In one embodiment, the environmentally benign solvent comprises acetic acid.

In one embodiment, the oxygen-containing gas comprises an oxygen content that is from 20.5 vol % to 100 vol %.

In one embodiment, the step of oxidizing is performed at a pressure that is from 0.5 bar to 10 bar.

In one embodiment, the step of oxidizing is performed at a temperature that is no more than 200° C.

In one embodiment, the step of oxidizing comprises adding from 0.10 mol % to 99.9 mol % of the benzenoid-based polymer source with respect to the reaction mixture.

In one embodiment, the step of oxidizing comprises adding from 1 molar equivalent to 10 molar equivalent of NOₓ source with respect to the number of repeating units present in the benzenoid-based polymer.

In one embodiment, the step of oxidizing is performed in the presence of from 0 mol % to 20 mol % of the catalyst with respect to the reaction mixture.

In one embodiment, the step of oxidizing is performed in the presence of from 0 mol % to 99.9 mol % of the environmentally benign solvent with respect to the reaction mixture.

In one embodiment, the NOₓ source is mixed with the solvent in a molar ratio of from 1:1 to 1:20.

In one embodiment, the yield of the oxidation product(s) is modulated by adding the NOₓ source at different time points.

In one embodiment, the benzenoid-based polymer source comprises waste polystyrene.

### Definitions

The term “polymer” as used herein refer to a chemical compound comprising repeating units and is created through a process of polymerization. It will be appreciated that the term “polymerization” is not limited to homopolymerization but also encompasses copolymerization. The units composing the polymer are typically derived from monomers and/or macromonomers. A polymer or copolymer typically comprises repetition of a number of constitutional units. The term “polymer” encompasses the term “homopolymer”, where the polymer is derived from a single type of monomer or homomonomer (or contains a single type of repeating unit). The term “polymer” also encompasses the term “copolymer”, where the polymer is derived from two or more different types of monomers or comonomers (or contains two or more different types of repeating units).

The terms “benzenoid” or “benzenoid ring” as used herein are to be interpreted broadly to refer to a substance or a compound which contains at least one benzene ring(s) in its structure. The benzenoid or benzenoid ring may comprise one or more benzene rings (e.g., 1, 2, 3, 4, 5, 6, 7, 8, 9, or 10) in its structure.

The term “NOₓ” as used herein is to be interpreted broadly to refer to any species (e.g., molecule, compound or ion) that comprise both nitrogen (N) and oxygen (O) atoms. Accordingly, the term “NOₓ” may include NOₓ existing in compound form such as N₂O (nitrous oxide), NO (nitric oxide), N₂O₂ (dinitrogen dioxide), N₂O₃ (dinitrogen trioxide), NO₂ (nitrogen dioxide), N₂O₄ (dinitrogen tetroxide) and N₂O₅ (dinitrogen pentoxide). The term “NOₓ” may also include NOₓ existing in ion/ionic form such as nitrite ion (NO₂⁻) and nitrate ion (NO₃⁻).

In the definitions of a number of substituents below, it is stated that “the group may be a terminal group or a bridging group”. This is intended to signify that the use of the term is intended to encompass the situation where the group is a terminal group/moiety as well as the situation where the group is a linker between two other portions of the molecule. Using the term “alkyl” having 1 carbon atom as an example, it will be appreciated that when existing as a terminal group, the term “alkyl” having 1 carbon atom may mean —CH₃ and when existing as a bridging group, the term “alkyl” having 1 carbon atom may mean —CH₂— or the like.

The term “alkyl” as a group or part of a group refers to a straight or branched aliphatic hydrocarbon group having 1 to 20 carbon atoms, 1 to 10 carbon atoms, 1 to 6 carbon atoms, or 1, 2, 3, 4, 5, 6, 7, 8, 9, 10, 11, 12, 13, 14, 15, 16, 17, 18, 19 or 20 carbon atoms. Examples of suitable straight and branched alkyl substituents include methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl and the like. The group may be a terminal group or a bridging group.

The term “alkenyl” as a group or part of a group denotes an aliphatic hydrocarbon group containing at least one carbon-carbon double bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of double bonds and the orientation about each double bond is independently E or Z. Exemplary alkenyl groups include, but are not limited to, ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 2-octenyl, 3-octenyl, 1-nonenyl, 2-nonenyl, 3-nonenyl, 1-decenyl, 2-decenyl, 3-decenyl and the like. The group may be a terminal group or a bridging group.

The term “alkynyl” as a group or part of a group denotes an aliphatic hydrocarbon group containing at least one carbon-carbon triple bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of triple bonds. Exemplary alkynyl groups include, but are not limited to, acetylenyl, propynyl, 1-butynyl, 2-butynyl, 3-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl and the like. The group may be a terminal group or a bridging group.

The term “aryl” as a group or part of a group denotes an optionally substituted monocyclic, or fused polycyclic, aromatic carbocycle (ring structure having ring atoms that are all carbon) preferably having from 5 to 20, or 5, 6, 7, 8, 9, 10, 11, 12, 13, 14, 15, 16, 17, 18, 19 or 20 carbon atoms per ring. Examples of aryl groups include but are not limited to phenyl, tolyl, xylyl, naphthyl, anthracenyl, phenanthrenyl, fluorenyl, indenyl or indanyl and the like.

The term “heteroaryl” as a group or part of a group refers to groups containing an aromatic ring (preferably a 5- or 6-membered aromatic ring) having one or more carbon atoms (for example 1 to 6 carbon atoms) in the ring replaced by a heteroatom. Suitable heteroatoms may include nitrogen (N) or (NH), oxygen (O) and sulfur(S). Examples of heteroaryl include but are not limited to thiophene, benzothiophene, benzofuran, benzimidazole, benzoxazole, benzothiazole, benzisothiazole, naphtha [2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenantridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl and the like. The group may be a terminal group or a bridging group.

The term “halogen” represents chlorine, fluorine, bromine or iodine. The term “halide” represents chloride, fluoride, bromide or iodide.

The term “optionally substituted,” when used to describe a chemical structure or moiety, refers to the chemical structure or moiety wherein one or more of its hydrogen atoms is optionally substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

The term “micro” as used herein is to be interpreted broadly to include dimensions from about 1 micron to about 1000 microns, about 1 micron to less than about 1000 microns, about 1 micron to about 900 microns, about 1 micron to about 800 microns, about 1 micron to about 700 microns, about 1 micron to about 600 microns, about 1 micron to about 500 microns, about 1 micron to about 400 microns, about 1 micron to about 300 microns, about 1 micron to about 200 microns, or from about 1 micron to about 100 microns.

The term “nano” as used herein is to be interpreted broadly to include dimensions in a nanoscale, i.e., less than about 1000 nm, about 1 nm to less than about 1000 nm, about 1 nm to about 900 nm, about 1 nm to about 800 nm, about 1 nm to about 700 nm, about 1 nm to about 600 nm, about 1 nm to about 500 nm, about 1 nm to about 400 nm, about 1 nm to about 300 nm, about 1 nm to about 200 nm, or from about 1 nm to about 100 nm. Accordingly, the term “nanostructures”, “nanoparticles”, “nanomaterials” and the like as used herein may include structures that have at least one dimension in the range of no more than said range. The term “nanostructures”, “nanoparticles”, “nanomaterials” and the like as used herein may include structures that have at least one dimension that is no more than about 100 nm, no more than about 90 nm, no more than about 80 nm, no more than about 70 nm, no more than about 60 nm, no more than about 50 nm, no more than about 40 nm, no more than about 30 nm, no more than about 20 nm, or no more than about 10 nm.

The term “nanostructure” as used herein broadly refers to an arrangement of interrelated elements in a system having at least one dimension in the nanoscale. The nanostructure described herein can include a nanoparticle, nanorod, nanofiber, nanoneedle, nanoplate, nanotube, and the like. The term “size” when used in the context of nanoparticle can refer to the diameter of the nanoparticle although it is not limited as such.

The term “particle” as used herein broadly refers to a discrete entity or a discrete body. The particle described herein can include an organic, an inorganic, a composite particle or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

It will also be appreciated that where priority is claimed to an earlier application, the full contents of the earlier application is also taken to form part of the present disclosure and may serve as support for embodiments disclosed herein.

### DESCRIPTION OF EMBODIMENTS

Exemplary, non-limiting embodiments of a method of converting a polymer comprising benzenoid rings into oxidation product(s) are disclosed hereinafter.

There is provided a method of converting a polymer comprising benzenoid rings (or benzenoid-based polymer) into oxidation/degradation/decomposition product(s). In various embodiments, the term “benzenoid-based polymer” may be used interchangeably with the term “benzenoid polymer”. The polymer comprising benzenoid rings may be derived from a benzenoid-based polymer containing source which includes but is not limited to benzenoid-based polymer waste, benzenoid-based polymer-based waste, benzenoid-based polymer waste, waste benzenoid-based polymer, unwanted benzenoid-based polymer or the like. In various embodiments, the method comprises recycling/upcycling (e.g., chemical recycling/upcycling) waste benzenoid-based polymer into valuable products/chemicals. Advantageously, in various embodiments, the method disclosed herein allows waste benzenoid-based polymer to be recycled/upcycled/converted directly into forms that are useful on their own or useful as feedstocks for further applications.

In various embodiments, the benzenoid-based polymer containing source is provided in the form of a solid, e.g., in the form of an article. The benzenoid-based polymer containing source may be a packaging material (such as protective/food packaging, containers, lids, beads, bottles, tray, disposable cutlery, take-away food boxes, bubble-tea cups etc.), foam, sheet, building/construction material, electronic part/structure, expanded polystyrene (EPS), extruded polystyrene (XPS), and styrofoam etc. The benzenoid-based polymer containing source may also comprise additives, chemicals, impurities and/or contaminants such as dyes, plasticizers, pigments, colouring agents, printing inks, fillers such as titanium oxide or zinc oxide, flame retardants, lubricants, adhesives, paper, cellulose material or the like or combinations thereof, which may be present in amounts of at least about 1 wt %, at least about 5 wt %, at least about 10 wt %, at least about 15 wt %, at least about 20 wt %, at least about 25 wt %, or at least about 30 wt %. Advantageously, in various embodiments, the benzenoid-based polymer source does not require pre-treatment, e.g., chemical/physical steps to remove additives, chemicals, impurities and/or contaminants (e.g., food waste) present in the benzenoid-based polymer source. Even more advantageously, the presence of additives, chemicals, impurities and/or contaminants in the benzenoid-based polymer source do not substantially prevent/deter the method from working efficiently and/or effectively. In various embodiments, the method disclosed herein allows the benzenoid-based polymer source to be recycled/upcycled/converted successfully into oxidation products, even in the presence of additives, chemicals, impurities and/or contaminants. In various embodiments, the method is minimally affected by presence of additives and dyes. In various embodiments therefore, the method is substantially devoid of a pre-treatment step to remove additives, chemicals, impurities and/or contaminants (e.g., food waste) from the benzenoid-based polymer containing source. It will, however, be appreciated that the benzenoid-based polymer containing source may be inspected for non-benzenoid polymer components/parts/contaminants and processed to remove such components/parts/contaminants prior to use. For example, a benzenoid-based polymer-based packaging may be inspected and processed to remove parts such as food waste before use.

In various embodiments, the method comprises a step of chemically converting/recycling/upcycling a benzenoid-based polymer containing source. In various embodiments, the method or the step of converting the polymer comprising benzenoid rings into oxidation/degradation product(s) comprises cleavage of the polymeric chains present in the benzenoid-based polymer into shorter/smaller/simpler chains. In various embodiments, the step of chemically treating/converting/recycling/upcycling comprises oxidation and/or degradation reaction. In various embodiments, the method comprises a step of oxidizing/degrading a benzenoid-based polymer source under suitable conditions to oxidize/degrade the benzenoid-based polymer. The oxidation/degradation reaction may be a partial oxidation/degradation or a complete oxidation/degradation. In various embodiments, the method or the step of converting the polymer comprising benzenoid rings into oxidation/degradation product(s) comprises one or more of the following reactions: H-abstraction, hydrogen atom transfer, beta-scission, O₂ combination, NO₂ combination, homolytic cleavage, chain continue reaction (e.g., initiation, propagation, branching, inhibition, termination etc), reactions involving reactive intermediates (e.g., free radicals), oxidation and the like and combinations thereof.

In various embodiments, the term “polymer comprising benzenoid rings” encompasses the terms “homopolymer comprising benzenoid rings” and “copolymer comprising benzenoid rings”. Accordingly, the term “benzenoid-based polymer” also encompasses the terms “benzenoid-based homopolymer” and “benzenoid-based copolymer”. In some embodiments, the benzenoid-based polymer comprises/consists essentially/consists of one or more benzene rings in all of its repeating units or in each of its repeating units. In some embodiments, the benzenoid-based polymer is devoid of repeating units/monomeric units that do not contain a benzene ring, e.g. polystyrene.

In various embodiments, the polymer comprising benzenoid rings (or benzenoid-based polymer) is derived from:
(M1) one or more benzenoid monomer(s); and
(M2) optionally one or more non-benzenoid monomer(s).

In various embodiments, the benzenoid monomer (M1) comprises one or more benzenoid ring(s). In various embodiments, the non-benzenoid monomer (M2) is substantially devoid of benzenoid ring. In various embodiments, benzenoid monomer (M1) comprises one or more polymerizable unsaturated bond(s). In various embodiments, non-benzenoid monomer (M2) comprises one or more polymerizable unsaturated bond(s). Examples of the polymerizable unsaturated bonds may include but are not limited to C═C, C≡C, the like, and combinations thereof.

In various embodiments, the benzenoid monomer (M1) comprises a structure that is represented by general formula (21):

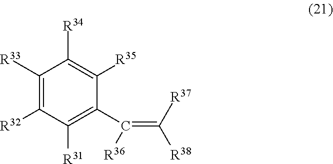

wherein
R³¹ to R³⁵ are each independently selected from the group consisting of H, optionally substituted alkyl, optionally substituted alkenyl, optionally substituted alkynyl, SO₃H, and halogens atoms (i.e. I, Cl, Br, F);
R³⁶ is H; and
R³⁷ to R³⁸ are each independently selected from the group consisting of H, optionally substituted alkyl, optionally substituted alkenyl and optionally substituted alkynyl.

In various embodiments, the polymer comprising benzenoid rings (or benzenoid-based polymer) is derived from a single type of monomer selected from one or more benzenoid monomers (M1). In such embodiments, the polymer comprising benzenoid rings (or benzenoid-based polymer) comprises a homopolymer. For example, the single type of benzenoid monomer may be styrene, which undergoes homopolymerization to obtain polystyrene.

In various embodiments, the polymer comprising benzenoid rings (or benzenoid-based polymer) is derived from two or more different types of monomers selected from one or more benzenoid monomers (M1). In various embodiments, the polymer comprising benzenoid rings (or benzenoid-based polymer) is derived from two or more different types of monomers selected from the group consisting of one or more benzenoid monomers (M1) and one or more non-benzenoid monomers (M2). In such embodiments, the polymer comprising benzenoid rings (or benzenoid-based polymer) comprises a copolymer. For example, the copolymer may be derived from two different types of monomers selected from (M1) and (M2) namely, styrene and butadiene, which undergo copolymerization to obtain poly(styrene-co-butadiene).

In various embodiments, the polymer comprising benzenoid rings (or the benzenoid-based polymer) comprises/contains:
(R1) one or more benzenoid repeating units represented by general formula (1A); and
(R2) optionally one or more non-benzenoid repeating units:

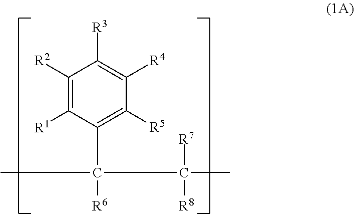

wherein
R¹ to R⁵ are each independently selected from the group consisting of H, optionally substituted alkyl, optionally substituted alkenyl, optionally substituted alkynyl, SO₃H, and halogens atoms (i.e. I, Cl, Br, F);
R⁶ is H; and
R⁷ to R⁸ are each independently selected from the group consisting of H, optionally substituted alkyl, optionally substituted alkenyl and optionally substituted alkynyl.

In various embodiments, the non-benzenoid repeating unit (R2) is substantially devoid of benzenoid ring. In various embodiments, the non-benzenoid repeating unit (R2) is represented by general formula (1B):

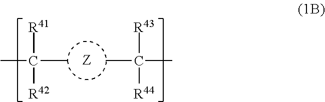

wherein
Z is optionally present as an organic group selected from the group consisting of optionally substituted alkyl, optionally substituted alkenyl, optionally substituted alkynyl, optionally substituted cycloalkyl, optionally substituted cycloalkenyl, and combinations thereof;
R⁴¹ to R⁴⁴ are each independently selected from the group consisting of hydrogen (—H), nitro (—NO₂), carboxylic acid (—COOH), cyano (—CN), ester (—COOR), halogen (e.g., —F, —CI, —Br, —I), hydroxy (—OH), alkoxy (—OR), sulfonic acid (—SO₃H), optionally substituted alkyl, optionally substituted alkenyl, optionally substituted alkynyl, optionally substituted cycloalkyl, optionally substituted cycloalkenyl, optionally substituted heteroaryl, and combinations thereof, where R is an organic group. R may be an organic group which includes but is not limited to alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

In various embodiments, the non-benzenoid repeating unit (R2) is represented by general formula (1C), (1D), (1E), (1F) and/or (1G):

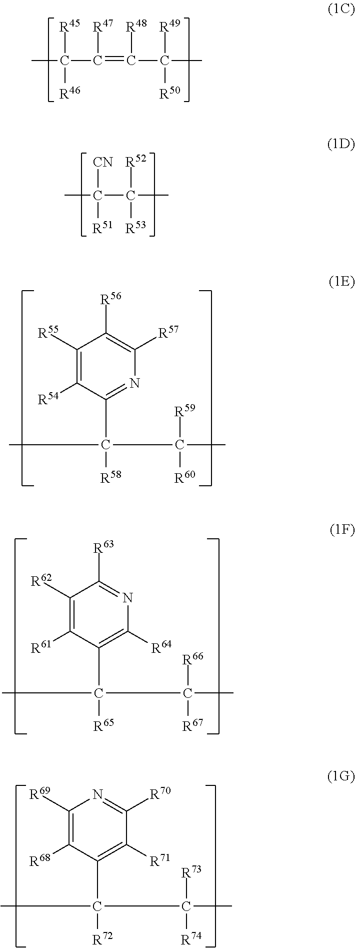

wherein R⁴⁵ to R⁷⁴ are each independently selected from the group consisting of H, optionally substituted alkyl, optionally substituted alkenyl and optionally substituted alkynyl.

In various embodiments, the repeating units contained in the benzenoid-based polymer are identical. In such embodiments, the benzenoid-based polymer comprises a homopolymer. The benzenoid-based polymer may comprise a homopolymer having a single type of repeating unit selected from benzenoid repeating units (R1) (e.g., derived from a single type of benzenoid monomer selected from one or more benzenoid monomers (M1)). In various embodiments, the homopolymer comprises benzenoid repeating units (R1), but not non-benzenoid repeating units (R2). Examples of “benzenoid-based homopolymer” includes but is not limited to, polystyrene, poly(2-methylstyrene), poly(3-methylstyrene), poly(4-methylstyrene), poly(2,4-dimethylstyrene), poly(2,5-dimethylstyrene) or the like.

In various embodiments, the repeating units contained in the benzenoid-based polymer are different. In such embodiments, the benzenoid-based polymer comprises a copolymer. In various embodiments, the copolymer comprises benzenoid repeating units (R1), but not non-benzenoid repeating units (R2). For example, the benzenoid-based polymer may comprise a copolymer comprising two or more different types of repeating unit selected from benzenoid repeating units (R1) (e.g., derived from two or more different types of benzenoid monomers selected from one or more benzenoid monomers (M1)). In various embodiments, the copolymer comprises benzenoid repeating units (R1) and non-benzenoid repeating units (R2). For example, the benzenoid-based polymer may comprise a copolymer comprising two or more different types of repeating unit selected from benzenoid repeating units (R1) and non-benzenoid repeating units (R2) (e.g., derived from two or more different types of benzenoid monomers selected from one or more benzenoid monomers (M1) and one or more non-benzenoid monomers (M2)). The benzenoid-based polymer may comprise a copolymer having two or more, three or more, four or more, or five or more different types of repeating unit. Examples of “benzenoid-based copolymer” includes but is not limited to, poly(styrene-co-butadiene), poly(styrene-co-acrylonitrile), poly(styrene-co-vinylpyridine) and the like.

In various embodiments, the polymer comprising benzenoid rings (or the benzenoid-based polymer) comprises:
(P1) homopolymer comprising identical repeating units;
(P2) copolymer comprising different repeating units that are chemically linked together;
(P3) two or more different (P1) that are not chemically linked together;
(P4) two or more different (P2) that are not chemically linked together; and
(P5) a combination comprising (P1), (P2), (P3), and/or (P4).

In various embodiments, the benzenoid-based polymer comprises (P1) homopolymer comprising identical repeating units represented by general formula (1A). In various embodiments, the homopolymer is derived from or obtained via polymerization of a single type of benzenoid monomer. For example, the benzenoid-based polymer may comprise a homopolymer having a structure represented by general formula (11):

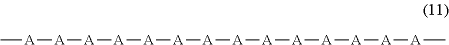

where A comprises a repeating unit represented by general formula (1A).

In various embodiments, the benzenoid-based polymer comprises (P2) copolymer comprising 2 or more different repeating units selected from benzenoid repeating units (R1); or selected from a mixture of benzenoid repeating units (R1) and non-benzenoid repeating units (R2) that are chemically linked together. In various embodiments, the copolymer is derived from or obtained via copolymerization of 2 or more, 3 or more, 4 or more, 5 or more, 6 or more, 7 or more, 8 or more, 9 or more, or 10 or more different types of benzenoid monomers or of a mixture of benzenoid and non-benzenoid monomers. In various embodiments, the copolymer comprises different types/segments that are chemically linked (e.g., covalently linked) together in the same polymer. The 2 or more segments may be joined as blocks in a regular/orderly/sequentially manner (e.g. as a block copolymer comprising repeating units arranged in AB sequence, ABA sequence or ABAB sequence, where A and B represent different types of repeating units), or may be randomly incorporated into/arranged within the polymer.

For example, the benzenoid-based polymer may comprise a copolymer having a structure represented by general formula (12), (13), (14) and/or (15):

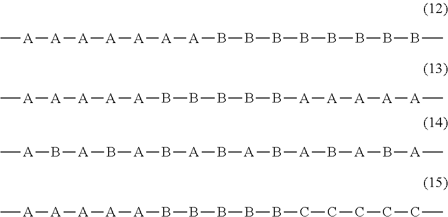

It should be appreciated that the arrangement of A, B and C may not follow the order shown in formula (12) to (15) above and they may be randomly or regularly arranged in any other manner.

In various embodiments, the benzenoid-based polymer comprises a copolymer having a structure with repeating units arranged in AB sequence. In some embodiments, A comprises a benzenoid repeating unit represented by general formula (1A); B comprises a benzenoid repeating unit represented by general formula (1A), and wherein A is different from B. In some embodiments, A comprises a benzenoid repeating unit represented by general formula (1A); and B comprises a non-benzenoid repeating unit, or vice versa. In various embodiments, the copolymer is a diblock copolymer.

In various embodiments, the benzenoid-based polymer comprises a copolymer having a structure with repeating units arranged in ABA sequence. In some embodiments, A comprises a benzenoid repeating unit represented by general formula (1A); B comprises a benzenoid repeating unit represented by general formula (1A), and wherein A is different from B. In some embodiments, A comprises a benzenoid repeating unit represented by general formula (1A); and B comprises a non-benzenoid repeating unit, or vice versa.

In various embodiments, the benzenoid-based polymer comprises a copolymer having a structure with repeating units arranged in ABAB sequence. In some embodiments, A comprises a benzenoid repeating unit represented by general formula (1A); B comprises a benzenoid repeating unit represented by general formula (1A), and wherein A is different from B. In some embodiments, A comprises a benzenoid repeating unit represented by general formula (1A); and B comprises a non-benzenoid repeating unit, or vice versa.

In various embodiments, the benzenoid-based polymer comprises a copolymer having a structure with repeating units arranged in ABC sequence, where A comprises a benzenoid or non-benzenoid repeating unit; B comprises a benzenoid or non-benzenoid repeating unit; C comprises a benzenoid or non-benzenoid repeating unit, and wherein A, B and C are all different from each other, and at least one of A, B and C comprises a benzenoid repeating unit represented by general formula (1A). In various embodiments, the copolymer is a triblock copolymer.

In various embodiments, the benzenoid-based polymer comprises (P3) two or more different (P1) homopolymers that are not chemically linked together. In various embodiments, the benzenoid-based polymer comprises a physical mixture/blend/composite having two or more individual benzenoid-based polymers that are not covalently joined together. For example, the benzenoid-based polymer may comprise a composition of two or more, three or more, four or more, or five or more different homopolymers that are physically mixed/blended together.

In various embodiments, the benzenoid-based polymer comprises (P4) two or more different (P2) copolymers that are not chemically linked together. In various embodiments, the benzenoid-based polymer comprises a physical mixture/blend/composite having two or more individual benzenoid copolymers that are not covalently joined together. For example, the benzenoid-based polymer may comprise a composition of two or more, three or more, four or more, or five or more different copolymers that are physically mixed/blended together.

In various embodiments, the benzenoid-based polymer comprises (P5) a combination comprising (P1), (P2), (P3) and/or (P4). For example, the benzenoid-based polymer may comprise (P1) and (P2); (P1) and (P3); (P1) and (P4); (P2) and (P3); (P2) and (P4); (P3) and (P4); (P2) and (P3) and (P4); (P1) and (P3) and (P4); (P1) and (P2) and (P4); (P1) and (P2) and (P3); or (P1) and (P2) and (P3) and (P4);

In various embodiments, the polymer comprising benzenoid rings (or the benzenoid-based polymer) is represented by general formula (1):

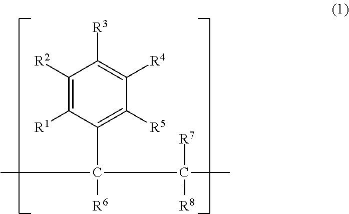

wherein
R¹ to R⁵ are each independently selected from the group consisting of H, optionally substituted alkyl, optionally substituted alkenyl, optionally substituted alkynyl, SO₃H, and halogens atoms (i.e. I, Cl, Br, F);
R⁶ is H;
R⁷ to R⁸ are each independently selected from the group consisting of H, optionally substituted alkyl, optionally substituted alkenyl and optionally substituted alkynyl; and
n≥1.

In various embodiments, n is from about 10 to about 5,000. For example, n may be a value from about 10 to about 5,000, from about 25 to about 4,750, from about 50 to about 4,500, from about 75 to about 4,250, from about 100 to about 4,000, from about 125 to about 3,750, from about 150 to about 3,500, from about 175 to about 3,250, from about 200 to about 3,000, from about 225 to about 2,750, from about 250 to about 2,500, from about 275 to about 2,250, from about 300 to about 2,000, from about 325 to about 1,750, from about 350 to about 1,500, from about 375 to about 1,250, from about 400 to about 1,000, from about 425 to about 975, from about 450 to about 950, from about 475 to about 925, from about 500 to about 900, from about 525 to about 875, from about 550 to about 850, from about 575 to about 825, from about 600 to about 800, from about 625 to about 775, from about 650 to about 750, from about 675 to about 725, or about 700.

In various embodiments, the benzenoid-based polymer comprises polystyrene. In various embodiments, the benzenoid-based polymer comprises unsubstituted polystyrene. In various embodiments, R¹, R², R³, R⁴, R⁵, R⁶, R⁷ and R⁸ are all H. In various embodiments, the benzenoid-based polymer comprises substituted polystyrene. In various embodiments, at least one of R¹, R², R³, R⁴ and R⁵ is/are not H. In various embodiments, at least one of R¹, R², R³, R⁴ and R⁵ is/are alkyl. In various embodiments, at least one of R¹, R², R³, R⁴ and R⁵ is/are CH₃. For example, the benzenoid-based polymer may be poly(2-methylstyrene), poly(3-methylstyrene), poly(4-methylstyrene), poly(2,4-dimethylstyrene), poly(2,5-dimethylstyrene), the like, or combinations thereof.

In various embodiments, the average molecular weight (M_{w}) of the benzenoid-based polymer is from about 1,000 to about 500,000, from about 2,500 to about 475,000, from about 5,000 to about 450,000, from about 7,500 to about 425,000, from about 10,000 to about 400,000, from about 25,000 to about 375,000, from about 50,000 to about 350,000, from about 75,000 to about 325,000, from about 100,000 to about 300,000, from about 125,000 to about 275,000, from about 150,000 to about 250,000, from about 175,000 to about 225,000, or about 200,000. For example, a commercial polymer comprising benzenoid rings having a molecular weight of from about 100,000 to about 400,000 (e.g., commercial polystyrene) may be used in embodiments of the method disclosed herein. A substituted polymer (e.g., polystyrene substituted with functional groups such as SO₃H, and/or halogens atoms), which may have a lower molecular weight (M_{w}) than a commercial polymer, may also be used in embodiments of the method disclosed herein.

In various embodiments, the average molecular mass (Mₙ) of the benzenoid-based polymer is from about 1,000 to about 500,000, from about 2,500 to about 475,000, from about 5,000 to about 450,000, from about 7,500 to about 425,000, from about 10,000 to about 400,000, from about 25,000 to about 375,000, from about 50,000 to about 350,000, from about 75,000 to about 325,000, from about 100,000 to about 300,000, from about 125,000 to about 275,000, from about 150,000 to about 250,000, from about 175,000 to about 225,000, or about 200,000. In various embodiments, the average molecular mass (Mₙ) of the benzenoid-based polymer is from about 1 kDa to about 50 kDa.

In various embodiments, the method comprises hydrogen abstraction as an initial step. In various embodiments, hydrogen abstraction (e.g., from R⁶ of general formula (1)) is allowed to take place before chain cleavage occurs on/at the benzenoid-based polymer. In various embodiments, the step of oxidizing begins with the removal of R⁶ from general formula (1).

In various embodiments, the step of oxidizing comprises conversion of one or more bonds in the benzenoid-based polymer containing source into carboxylic acid group (C(═O) OH), thereby introducing carboxylic acid groups into the benzenoid-based polymer. In various embodiments therefore, the oxidation/degradation product comprises an organic acid functional group. In various embodiments, the organic acid functional group comprises an oxygenated organic acid functional group such as carboxylic acid functional group (—COOH). The oxidation/degradation product may comprise one, two, three, four, five, six, seven, eight, nine or ten organic acid functional groups. In various embodiments, alkyl group(s) that is/are attached directly to benzene ring is/are converted into carboxylic acid groups. For example, a substituted benzenoid-based polymer comprising an alkyl group attached to the benzene ring (e.g., poly(4-methylstyrene)) may be converted into an oxidation/degradation product comprising additional carboxylic acid groups (e.g., terephthalic acid).

In various embodiments, the oxidation/degradation product(s) comprises one or more compounds represented by general formula (2):

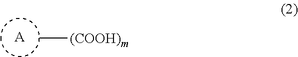

wherein
A is selected from the group consisting of H, optionally substituted alkyl, optionally substituted alkenyl, optionally substituted alkynyl, optionally substituted 5-membered ring and optionally substituted 6-membered ring; and
m≥1.

In various embodiments, m is from about 1 to about 6. For example, m may be 1, 2, 3, 4, 5 or 6. In various embodiments, the oxidation/degradation product(s) comprise monocarboxylic acid (e.g., m=1), dicarboxylic acid (e.g., m=2) and/or tricarboxylic acid (e.g., m=3)

In various embodiments, the compound represented by general formula (2) comprises an aliphatic carboxylic acid. In various embodiments, A is H. For example, the oxidation/degradation product may comprise formic acid or methanoic acid.

In various embodiments, A is optionally substituted alkyl. For example, the oxidation/degradation product may comprise linear alkyl monocarboxylic acid, dicarboxylic acid and/or tricarboxylic acid. In various embodiments where A is optionally substituted alkyl and m is 2, the oxidation/degradation product(s) comprises one or more compounds selected from general formula (2A):

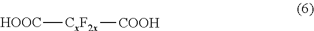

wherein
x≥1; and
one or more of hydrogen atom in CₓH₂ₓ is optionally substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

In various embodiments, x is ≥1, ≥2, ≥3, ≥4, ≥5, ≥6, ≥7, ≥8, ≥9, ≥10, ≥11, ≥12, ≥13, ≥14, ≥15, ≥16, ≥17, ≥18, ≥19, ≥20, ≥21, ≥22, ≥23, ≥24, ≥ 25, ≥26, ≥27, ≥28, ≥29, ≥30, ≥31, ≥32, ≥33, ≥34, ≥35, ≥36, ≥37, ≥38, ≥ 39, ≥40, ≥41, ≥42, ≥43, ≥44, ≥45, ≥46, ≥47, ≥48, ≥49, ≥50, ≥75 or ≥100.

In various embodiments, A is a 5-membered ring. For example, A may be selected from pyrrole, thiophene, furan, pyrazole, imidazole, imidazoline, oxazole, isoxazole, thiazole, isothiazole, triazole, oxadiazole, thiadiazole, tetrazole, the like or combinations thereof. It will be appreciated that any 5-membered ring that is resistant to oxidation/degradation or is not prone/susceptible to oxidation/degradation (e.g., is not quenched during the oxidation/degradation) may be present in embodiments of the oxidation/degradation product(s).

In various embodiments, A is optionally substituted 6-membered ring. For example, A may be an optionally substituted phenyl ring. In various embodiments where A is an optionally substituted phenyl ring, the oxidation/degradation product(s) comprises one or more compounds selected from general formula (2B):

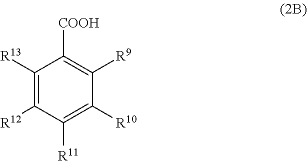

wherein
R⁹ to R¹³ are each independently selected from the group consisting of hydrogen (—H), nitro (—NO₂), carboxylic acid (—COOH), cyano (—CN), ester (—COOR), halogen (e.g., —F, —Cl, —Br, —I), hydroxy (—OH), alkoxy (—OR), sulfonic acid (—SO₃H), optionally substituted alkyl, optionally substituted alkenyl, and optionally substituted alkynyl, where R is an organic group. R may be an organic group which includes but is not limited to alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

In various embodiments, general formula (2B) comprises 1, 2, 3, 4, 5 or 6 carboxylic acid groups (—COOH). In various embodiments, the number of carboxylic acid groups present in general formula (2B) correspond to the number of alkyl, alkenyl and/or alkynyl groups that are present in a repeating unit of the benzenoid-based polymer represented by general formula (1). For example, when the benzenoid-based polymer contains two alkyl groups (e.g., primary) (1°) and/or secondary (2°) alkyl groups) attached to its benzenoid ring in a repeating unit, said two alkyl groups are eventually oxidized to carboxylic acids (e.g., the product final comprises terephthalic acid), thereby producing an oxidation/degradation product containing a total of two carboxylic acids. In various embodiments therefore, when the benzenoid units on the polymer starting material contain more than one alkyl, alkenyl and/or alkynyl groups, then oxidation/degradation produces benzenoid compounds with more than one COOH groups.

In various embodiments, the oxidation/degradation products comprise one or more of the following carboxylic acids: terephthalic acid, phthalic acid, isophthalic acid, benzoic acid, o-nitrobenzoic acid, m-nitrobenzoic acid, p-nitrobenzoic acid, formic acid and the like and combinations thereof.

In various embodiments, the step of oxidizing converts/oxidizes the polymer comprising benzenoid rings into oxidation/degradation product that comprises one or more simpler carboxylic acids selected from the group consisting of benzoic acid, p-nitrobenzoic acid, formic acid and the like and combinations thereof.

In various embodiments, the step of oxidizing a benzenoid-based polymer source comprises adding a NOₓ source to the benzenoid-based polymer source under suitable conditions to oxidize/degrade the benzenoid-based polymer. In various embodiments, the NOₓ source possesses strong oxidizing ability/strength and/or behaves/acts as a strong oxidizing agent or oxidant. Advantageously, the NOₓ source supplies/provides a source or pool of nitrite ions (NO₂⁻) and/or nitrate ions (NO₃⁻) for oxidation to take place. In various embodiments, the NOₓ source comprises a source that supplies nitrite ions (NO₂⁻) and/or nitrate ions (NO₃⁻). For example, nitrite ion (NO₂⁻) may be supplied/provided by nitrous acid (HNO₂) and/or nitrite salts, while nitrate ion (NO₃⁻) may be supplied/provided by nitric acid (HNO₃) and/or nitrate salts. In various embodiments, the NOₓ source is selected from the group consisting of nitrous acid (HNO₂), nitrite salts, nitric acid (HNO₃), nitrate salts and the like, and combinations thereof.

In various embodiments, the NOₓ source comprises an inorganic and/or organic nitrate salt. The inorganic/organic nitrate salt may be hydrated or anhydrous. In various embodiments, the NOₓ source comprises an inorganic nitrate salt such as a nitrate salt of a metal selected from lithium (Li), sodium (Na), potassium (K), rubidium (Rb), caesium (Cs), francium (Fr), beryllium (Be), magnesium (Mg), calcium (Ca), strontium (Sr), barium (Ba), radium (Ra), copper (Cu), zinc (Zn), nickel (Ni), cobalt (Co), iron (Fe), manganese (Mn), aluminium (Al), chromium (Cr), vanadium (V), titanium (Ti), scandium (Sc), yttrium (Y), zirconium (Zr), niobium (Nb), molybdenum (Mo), technetium (Tc), ruthenium (Ru), rhodium (Rh), palladium (Pd), silver (Ag), cadmium (Cd), lanthanide/lanthanoid (i.e. f block elements lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu)), hafnium (Hf), tantalum (Ta), tungsten (W), rhenium (Re), osmium (Os), iridium (Ir), platinum (Pt), gold (Au), mercury (Hg), actinium (Ac), or the like or combinations thereof. In various embodiments, the NOₓ source is selected from a metal nitrate which includes, but is not limited to sodium nitrate (NaNO₃), potassium nitrate (KNO₃), magnesium nitrate (Mg(NO₃)₂), calcium nitrate (Ca(NO₃)₂), barium nitrate (Ba(NO₃)₂), aluminium nitrate Al(NO₃)₃, copper nitrates (e.g., copper(II) nitrate Cu(NO₃)₂), zinc nitrate (Zn(NO₃)₂), tetra-n-butylammonium nitrate, cerium nitrates (e.g., cerium(III) nitrate Ce(NO₃)₃, cerium(IV) ammonium nitrate (CAN) (NH₄)₂[Ce(NO₃)₆]), iron nitrates (e.g., iron(III) nitrate Fe(NO₃)₃), lanthanide/lanthanoid (i.e. f block elements) nitrates (e.g., lanthanum nitrate (La(NO₃)₃)) and the like, and combinations thereof.

Advantageously, in various embodiments, the method does not require (direct or external) addition of gaseous harmful reactants such as nitrogen oxides e.g., NO (g), NO₂ (g), NO₃ (g), N₂O₄ (g), N₂O₅ (g) or the like or combinations thereof. Accordingly, in various embodiments, the NOₓ source is substantially devoid of gaseous nitrogen oxides such as NO (g), NO₂ (g), NO₃ (g), N₂O₄ (g), N₂O₅ (g) or the like or combinations thereof. It will be appreciated, however, that the reaction mixture may comprise gaseous nitrogen oxides (e.g., NO₂ and NO), which may be produced during thermal decomposition of the NOₓ source (e.g., decomposition of nitrate salts or nitric acid) as main reactive intermediates at a controlled rate.

In various embodiments, the NOₓ source is added at different time points, e.g. at a predetermined/regular/staggered time interval. For example, the NOₓ source may be added at about 1-minute time interval, about 5-minutes time intervals, about 10-minutes time intervals, about 15-minutes time intervals, about 20-minutes time intervals, about 25-minutes time intervals, about 30-minutes time intervals, about 35-minutes time intervals, about 40-minutes time intervals, about 45-minutes time intervals, about 50-minutes time intervals, about 55-minutes time intervals, about 1-hour time intervals, about 2-hours time intervals, about 3-hours time intervals, about 4-hours time intervals, about 5-hours time intervals, about 6-hours time intervals, about 7-hours time intervals, about 8-hours time intervals, about 9-hours time intervals, about 10-hours time intervals, about 11-hours time intervals, about 12-hours time intervals, about 13-hours time intervals, about 14-hours time intervals, about 15-hours time intervals, about 16-hours time intervals, about 17-hours time intervals, about 18-hours time intervals, about 19-hours time intervals, about 20-hours time intervals, about 21-hours time intervals, about 22-hours time intervals, about 23-hours time intervals, about 24-hours time intervals, about 25-hours time intervals, about 26-hours time intervals, about 27-hours time intervals, about 28-hours time intervals, about 29-hours time intervals, about 30-hours time intervals, about 31-hours time intervals, about 32-hours time intervals, about 33-hours time intervals, about 34-hours time intervals, about 35-hours time intervals, about 36-hours time intervals, about 37-hours time intervals, about 38-hours time intervals, about 39-hours time intervals, about 40-hours time intervals, about 41-hours time intervals, about 42-hours time intervals, about 43-hours time intervals, about 44-hours time intervals, about 45-hours time intervals, about 46-hours time intervals, about 47-hours time intervals, about 48-hours time intervals, about 60-hours time intervals, about 72-hours time intervals, about 84-hours time intervals, or about 96-hours time intervals. In various embodiments, the NOₓ source is added to the benzenoid-based polymer source for a total of 1 time, 2 times, 3 times, 4 times, 5 times, 6 times, 7 times, 8 times, 9 times, or 10 times during/throughout the entire duration of the oxidation/degradation reaction. For example, the NOₓ source may be added to the benzenoid-based polymer source twice every 24 hours (for a 48-hours reaction), or thrice every 24 hours (for a 72-hours reaction). Advantageously, adding the NOₓ source at different time points successfully converts benzenoid-based polymer into its oxidation/degradation products with a high conversion/production yield.

In various embodiments, the step of oxidizing/degradation is carried out in the presence of:
(i) an oxygen-containing gas;
(ii) optionally an environmentally benign solvent; and
(iii) optionally a catalyst.

In various embodiments, the step of oxidizing a benzenoid-based polymer source is performed in the presence of an oxidizing agent or oxidant such as an oxygen-containing gas. The oxygen-containing gas may be selected from air (e.g., atmospheric air or ambient air) or gas mixture comprising oxygen. The gas mixture may comprise oxygen content that is from about 20.5% to about 100.0 vol %, from about 21.0 vol % to about 100.0 vol %, from about 21.5 vol % to about 99.5 vol %, from about 22.0 vol % to about 99.0 vol %, from about 22.5 vol % to about 98.5 vol %, from about 23.0 vol % to about 98.0 vol %, from about 23.5 vol % to about 97.5 vol %, from about 24.0 vol % to about 97.0 vol %, from about 24.5 vol % to about 96.5 vol %, from about 25.0 vol % to about 96.0 vol %, from about 30.0 vol % to about 95.5 vol %, from about 35.0 vol % to about 95.0 vol %, from about 40.0 vol % to about 90.0 vol %, from about 45.0 vol % to about 85.0 vol %, from about 50.0 vol % to about 80.0 vol %, from about 55.0 vol % to about 75.0 vol %, from about 60.0 vol % to about 70.0 vol %, or about 65.0 vol %. In various embodiments, the oxygen-containing gas comprises about 100.0 vol % (i.e. pure oxygen), less than about 100.0 vol %, less than about 99.0 vol %, less than about 95.0 vol %, less than about 90.0 vol %, less than about 85.0 vol %, less than about 80.0 vol %, less than about 75.0 vol %, less than about 70.0 vol %, less than about 65.0 vol %, less than about 60.0 vol %, less than about 55.0 vol %, less than about 50.0 vol %, less than about 45.0 vol %, or less than about 40.0 vol % of oxygen.

For example, the oxygen-containing gas may comprise from about 5.0 vol % to about 35.0 vol %, from about 6.0 vol % to about 34.0 vol %, from about 7.0 vol % to about 33.0 vol %, from about 8.0 vol % to about 32.0 vol %, from about 9.0 vol % to about 31.0 vol %, from about 10.0 vol % to about 30.0 vol %, from about 11.0 vol % to about 29.0 vol %, from about 12.0 vol % to about 28.0 vol %, from about 13.0 vol % to about 27.0 vol %, from about 14.0 vol % to about 26.0 vol %, from about 15.0 vol % to about 25.0 vol %, from about 16.0 vol % to about 24.0 vol %, from about 17.0 vol % to about 23.0 vol %, from about 18.0 vol % to about 22.0 vol %, from about 19.0 vol % to about 21.0 vol %, from about 20.1 vol % to about 20.9 vol %, from about 20.2 vol % to about 20.8 vol %, from about 20.3 vol % to about 20.7 vol %, from about 20.4 vol % to about 20.6 vol %, or about 20.5 vol % of oxygen. It will be appreciated that there is no strict limit on the content of oxygen of the oxygen-containing gas used in embodiments of the method as long as the oxygen-containing gas is provided in excess. For example, even if the oxygen-containing gas comprises a low amount of oxygen (e.g., less than about 21.0 vol % of oxygen), the benzenoid-based polymer source may still be successfully converted into oxidation/degradation product (even though the reaction may be less efficient and/or longer reaction times may be required to achieve comparable yields).

In various embodiments, the step of oxidizing is carried out under atmospheric conditions (e.g., in open air). Advantageously, embodiments of the method allow benzenoid-based polymer to be recycled/upcycled/converted efficiently and/or effectively into oxidation products, without the need or requirement of a pure O₂ atmosphere.

In various embodiments, the step of oxidizing a benzenoid-based polymer source is performed in the presence of at least one of an environmentally benign solvent or a catalyst. In some embodiments, the step of oxidizing a benzenoid-based polymer source is performed in the presence of both an environmentally benign solvent and a catalyst.

In various embodiments, the step of oxidizing is performed in the presence of a green, environmentally friendly or environmentally benign solvent. In various embodiments, the solvent is one that does not become/get substantially oxidized (e.g., under the presently disclosed reaction conditions). It will be appreciated that, in various embodiments, it is not necessary for the polymer to be dissolved in the solvent (e.g., to be initially soluble). In various embodiments, the solvent provides a liquid bulk phase, which may or may not dissolve the polymer at any temperature. In various embodiments, the solvent is one that effectively serves as a medium or provides a liquid bulk phase to contain the reaction mixture (e.g., reaction mixture comprising the benzenoid-based polymer and NOₓ source). In various embodiments, the solvent comprises a weak acid or a weak organic acid. In various embodiments, the solvent comprises acetic acid (or ethanoic acid).

Advantageously, in various embodiments, the method does not require (direct or external) addition of toxic solvents such as aromatic solvents (e.g., benzene) or chlorinated solvents (e.g., 1,2-dichloroethane or dichloromethane) for dissolving the reaction mixture, thereby making the method green, environmentally sustainable and/or environmentally friendly. Accordingly, in various embodiments, the step of oxidizing is substantially devoid of aromatic and chlorinated solvents.

In various embodiments, the step of oxidizing is performed in the presence of a catalyst. Advantageously, the presence of a catalyst aids in promoting/accelerating the oxidation of the benzenoid-based polymer. In various embodiments, the catalyst comprises one or more N-hydroxyl (—N—OH) groups. Accordingly, in various embodiments, the step of oxidizing/degrading is performed in the presence of a N-hydroxyl catalyst or a catalyst comprising N-hydroxyl group(s).

In various embodiments, the N-hydroxyl group in the catalyst is arranged such that the N atom forms part of or is contained in a cyclic/ring moiety (e.g., in a 5-membered or 6-membered ring). In various embodiments, the catalyst comprises a structure selected from general formula (3), (4) and/or (5):

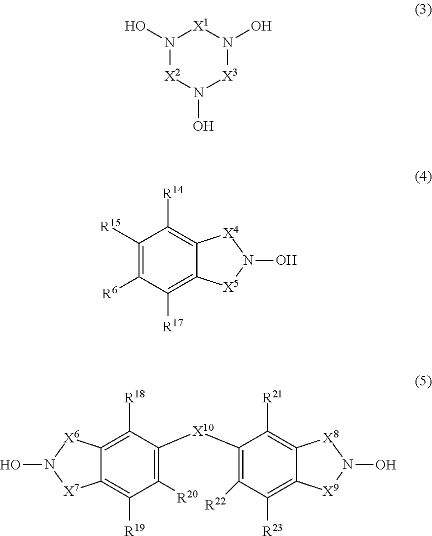

wherein
X¹ to X¹⁰ each independently selected from the group consisting of CH₂, C(═O), S(═O)₂ and combinations thereof; and
R¹⁴ to R²³ are each independently selected from the group consisting of hydrogen (—H), nitro (—NO₂), carboxylic acid (—COOH), cyano (—CN), ester (—COOR), amine (—NR₂), halogen (e.g., —F, —CI, —Br, —I), hydroxy (—OH), alkoxy (—OR), optionally substituted alkyl, optionally substituted alkenyl, and optionally substituted alkynyl, where R is an organic group. R may be an organic group which includes but is not limited to alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

In various embodiments, the catalyst comprises one or more structures selected from general formula (6), (7), (8) and/or (9):

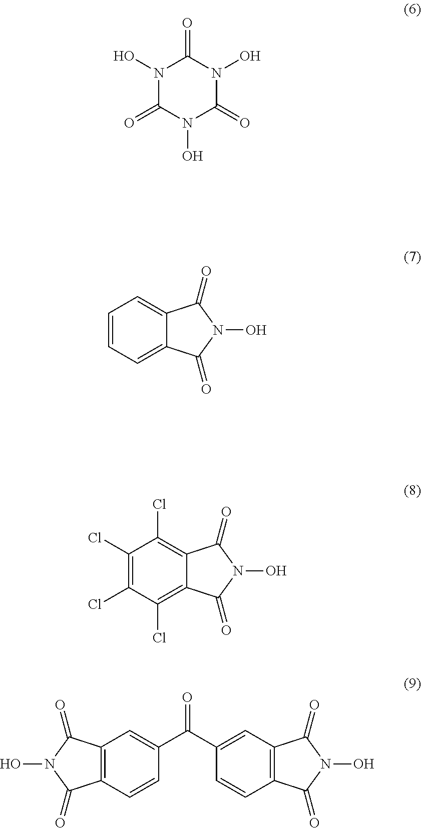

In various embodiments, the catalyst is selected from the group consisting of N-hydroxyphthalimide (NHPI) and derivatives thereof. In various embodiments, derivatives of NHPI comprises NHPI bearing/having different functional groups on the aromatic ring such as N-Hydroxytetrachlorophthalimide (Cl₄NHPI)), N,N′,N″-trihydroxyisocyanuric acid (THICA) and combinations thereof.

In various embodiments, the catalyst comprises one, two, three, four, five or six N-hydroxyl (—N—OH) groups. In embodiments where the catalyst comprises a structure represented by general formula (4) (e.g., N-hydroxyphthalimide (NHPI)), the total number of N-hydroxyl (—N—OH) group in the structure of said catalyst is 1. In embodiments where the catalyst comprises a structure represented by general formula (3) (e.g., N,N′,N″-trihydroxyisocyanuric acid (THICA) moiety), the total number of N-hydroxyl (—N—OH) groups in the structure of said catalyst is 3. In various embodiments, the total number of N-hydroxyl (—N—OH) groups present in the catalyst is dependent on the total number of the structures represented by general formula (3), (4) and/or (5) that are present in the catalyst. For example, a catalyst that comprises a polymer support containing 4 structures represented by general formula (4) would comprise a total number of 4-hydroxyl (—N—OH) groups in the catalyst.

In various embodiments, the step of oxidizing is substantially devoid of the use of metal in the catalyst. Advantageously, the use of a non-metal catalyst avoids/minimizes the problems of metal waste generation and/or disposal, and possibility of causing metal pollution in the environment, thereby making the method green, environmentally sustainable and/or environmentally friendly. In various embodiments, the catalyst is a metal-free catalyst or is substantially devoid of the presence of metal (e.g., transition metal such as Co, Mn, Fe, Zr etc).

In various embodiments, the step of oxidizing is performed in the presence of an acid. The acid may be inorganic/mineral acid such as hydrochloric acid (HCl), sulfuric acid (H₂SO₄) and boric acid. The acid may also be organic acid (e.g., strong organic acid) such as trifluoroacetic acid (TFA).

In various embodiments, the step of oxidizing is performed/carried out under heterogeneous conditions. The step of oxidizing/degrading may be performed in a heterogenous mixture that comprises two or more immiscible components/phases. For example, the immiscible components may be the benzenoid-based polymer source and the solvent. Advantageously, in various embodiments, the reaction works effectively under heterogeneous conditions and does not require the benzenoid-based polymer/polymer source to be soluble or substantially soluble in the solvent at the start of the reaction.

In various embodiments, the step of oxidizing is performed at a pressure that is from about 0.5 atm/bar to about 10.0 atm/bar, from about 0.6 atm/bar to about 9.5 atm/bar, from about 0.7 atm/bar to about 9.0 atm/bar, from about 0.8 atm/bar to about 8.5 atm/bar, from about 0.9 atm/bar to about 8.0 atm/bar, from about 1.0 atm/bar to about 7.5 atm/bar, from about 1.1 atm/bar to about 7.0 atm/bar, from about 1.2 atm/bar to about 6.5 atm/bar, from about 1.3 atm/bar to about 6.0 atm/bar, from about 1.4 atm/bar to about 5.5 atm/bar, from about 1.5 atm/bar to about 5.0 atm/bar, from about 1.6 atm/bar to about 4.5 atm/bar, from about 1.7 atm/bar to about 4.0 atm/bar, from about 1.8 atm/bar to about 3.5 atm/bar, from about 1.9 atm/bar to about 3.0 atm/bar, from about 2.0 atm/bar to about 2.9 atm/bar, from about 2.1 atm/bar to about 2.8 atm/bar, from about 2.2 atm/bar to about 2.7 atm/bar, from about 2.3 atm/bar to about 2.6 atm/bar, from about 2.4 atm/bar to about 2.5 atm/bar, or about 2.45 atm/bar. In various embodiments, an increased efficiency is achieved with higher pressure (e.g., at a pressure of 1 atm or more than about 1 atm). It will be appreciated, however, that even if a low pressure is employed/applied during the oxidation step (e.g., less than about 1 atm), the benzenoid-based polymer source may still be successfully converted into oxidation/degradation product (even though the reaction may be less efficient/effective).

In various embodiments, the method or oxidizing step is substantially devoid of a step that is performed at a pressure that is more than about 1 atm/bar. For example, the step of oxidizing/degrading may be performed at a pressure that is no more than about 5 atm/bar, no more than about 4 atm/bar, no more than about 3 atm/bar, no more than about 2 atm/bar, or no more than about 1 atm/bar.

In various embodiments, the step of oxidizing is performed at a temperature that is at least about 80.0° C., at least about 85.0° C., at least about 90.0° C., at least about 95.0° C., at least about 96.0° C., at least about 97.0° C., at least about 98.0° C., at least about 99.0° C., at least about 100.0° C., at least about 101.0° C., at least about 102.0° C., at least about 103.0° C., at least about 104.0° C., at least about 105.0° C., at least about 106.0° C., at least about 107.0° C., at least about 108.0° C., at least about 109.0° C., at least about 110.0° C., at least about 111.0° C., at least about 112.0° C., at least about 113.0° C., at least about 114.0° C., at least about 115.0° C., at least about 116.0° C., at least about 117.0° C., at least about 118.0° C., at least about 119.0° C., at least about 120.0° C., at least about 130.0° C., at least about 140.0° C., at least about 150.0° C., from about 80.0° C. to about 150.0° C., from about 80.0° C. to about 140.0° C., from about 80.0° C. to about 130.0° C., from about 80.0° C. to about 120.0° C., from about 85.0° C. to about 119.0° C., from about 90.0° C. to about 118.0° C., from about 95.0° C. to about 117.0° C., from about 96.0° C. to about 116.0° C., from about 97.0° C. to about 115.0° C., from about 98.0° C. to about 114.0° C., from about 99.0° C. to about 113.0° C., from about 100.0° C. to about 112.0° C., from about 101.0° C. to about 111.0° C., from about 102.0° C. to about 110.0° C., from about 103.0° C. to about 109.0° C., from about 104.0° C. to about 108.0° C., from about 105.0° C. to about 107.0° C., or about 106.0° C. In various embodiments, the oxidation/degradation reaction comprises thermal oxidation/degradation. It will be appreciated that there is no strict upper limit on the temperature employed/applied during the oxidation step. In various embodiments, the efficiency of the method/oxidation step increases with increasing temperature.

In various embodiments, the method or oxidizing step is substantially devoid of a step that is performed at a temperature that is more than about 120° C. For example, the step of oxidizing/degrading may be performed at a temperature that is no more than about 200° C., no more than about 150° C., no more than about 140° C., no more than about 130° C., or no more than about 120° C. In various embodiments, the method or oxidizing step is substantially devoid of or independent of use of light irradiation/penetration. Advantageously, embodiments of the method allows benzenoid-based polymer to be recycled/upcycled/converted into oxidation products, without the need for expensive and/or tedious/complex/sophisticated/harsh chemical upcycling/recycling/recovering techniques that require high energy input (e.g., pyrolysis etc) or light (e.g., photoreactions).

In various embodiments, the oxidizing step is performed over a time duration of at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 36 hours, at least about 48 hours, at least about 60 hours, at least about 72 hours, at least about 84 hours, at least about 96 hours, at least about 108 hours, at least about 120 hours, at least about 132 hours, at least about 144 hours, at least about 156 hours, at least about 168 hours, at least about 180 hours, at least about 192 hours, at least about 204 hours, at least about 216 hours, at least about 228 hours, at least about 240 hours, from about 1 hour to about 240 hours, from about 2 hours to about 228 hours, from about 3 hours to about 216 hours, from about 4 hours to about 204 hours, from about 5 hours to about 192 hours, from about 6 hours to about 180 hours, from about 7 hours to about 168 hours, from about 8 hours to about 156 hours, from about 9 hours to about 144 hours, from about 10 hours to about 132 hours, from about 11 hours to about 120 hours, from about 12 hours to about 108 hours, from about 13 hours to about 96 hours, from about 14 hours to about 84 hours, from about 15 hours to about 72 hours, from about 16 hours to about 60 hours, from about 17 hours to about 48 hours, from about 18 hours to about 36 hours, about 19 hours, about 20 hours, about 21 hours, about 22 hours, about 23 hours, or about 24 hours. It will be appreciated that in some embodiments, product yields do not increase proportionately with reaction times.

In various embodiments, the method further comprises, prior to the oxidizing/degrading step, a step of incubating the mixture comprising benzenoid-based polymer source and NOₓ source, in the presence of an oxygen-containing gas. The incubating step may be performed over a time duration of about 5 minutes, about 10 minutes, about 15 minutes, about 45 minutes, about 30 minutes, about 1 hour, about 2 hours, or about 3 hours. The incubating step may be performed at a temperature that is at least about 80.0° C., at least about 85.0° C., at least about 90.0° C., at least about 95.0° C., at least about 96.0° C., at least about 97.0° C., at least about 98.0° C., at least about 99.0° C., at least about 100.0° C., at least about 101.0° C., at least about 102.0° C., at least about 103.0° C., at least about 104.0° C., at least about 105.0° C., at least about 106.0° C., at least about 107.0° C., at least about 108.0° C., at least about 109.0° C., at least about 110.0° C., at least about 111.0° C., at least about 112.0° C., at least about 113.0° C., at least about 114.0° C., at least about 115.0° C., at least about 116.0° C., at least about 117.0° C., at least about 118.0° C., at least about 119.0° C., at least about 120.0° C., at least about 130.0° C., at least about 140.0° C., or at least about 150.0° C. The incubation step may be performed at a pressure that is at least about 0.5 atm/bar, at least about 0.6 atm/bar, at least about 0.7 atm/bar, at least about 0.8 atm/bar, at least about 0.9 atm/bar, at least about 1.0 atm/bar, at least about 1.1 atm/bar, at least about 1.2 atm/bar, at least about 1.3 atm/bar, at least about 1.4 atm/bar, at least about 1.5 atm/bar, at least about 1.6 atm/bar at least about 1.7 atm/bar, at least about 1.8 atm/bar, at least about 1.9 atm/bar, or at least about 2.0 atm/bar. In various embodiments, there is an incubation period of about 3 hours. The incubation may be performed at about 120.0° C. and at about 1 atm in the presence of an oxygen-containing gas. The incubation period/time/duration may be shortened by increasing the temperature and/or pressure employed/applied.

Advantageously, in various embodiments, the method is substantially devoid of/does not require a pre-treatment/pre-processing step to convert the benzenoid-based polymer source into powder/dust/particle form via physical/mechanical means (e.g., powderization, pulverization, grinding and/or crushing), prior to the oxidizing/degrading step. While it should be appreciated that such pre-treatment/pre-processing are not strictly required in various embodiments of the method, it is still possible to still carry out such steps to increase the surface area of the benzenoid-based polymer for reaction, which can increase the degradation speed.

In various embodiments, the step of oxidizing/degrading is designed to be customizable/tunable to obtain the desired oxidation/degradation product(s). For example, the reaction conditions employed for the step of oxidizing/degrading can be designed/adjusted to obtain a single specific product (e.g., purely BA or purely NBA). The reaction conditions employed for the step of oxidizing/degrading can also be designed/adjusted to obtain a mixture of products (e.g., BA and NBA).

In various embodiments, the ratio of different oxidation/degradation products obtained is customizable/tunable to a desired ratio by adjusting one or more of the following:
(1) number of components in the reaction mixture;
(2) type/choice of components in the reaction mixture;
(3) concentration/amount of components in the reaction mixture;
(4) temperature employed during oxidizing/degrading step;
(5) pressure employed during oxidizing/degrading step; and
(6) duration of oxidizing/degrading step.

Advantageously, in various embodiments, the method is designed such that the ratio of different oxidation/degradation products (e.g., benzoic acid (BA) to nitrobenzoic acid (NBA)) is customizable/tunable/controllable/adjustable, depending on the desired ratio to be achieved. For example, the ratio of BA:NBA may be adjusted by changing identity of the NOₓ source (or oxidizing strength of the NOₓ source). The ratio of BA:NBA may also be adjusted by removing/adding a solvent and/or a catalyst from/to the reaction mixture. The ratio of BA:NBA may also be adjusted by using a higher/lower temperature for the oxidizing/degrading step. In various embodiments, the method is designed such that the oxidation/degradation product comprises a mixture of benzoic acid (BA) and nitrobenzoic acid (NBA). The nitrobenzoic acid may be 2-nitrobenzoic acid, 3-nitrobenzoic acid or 4-nitrobenzoic acid. In other embodiments, the method is designed such that the oxidation/degradation product comprises a single isomer. For example when the oxidation/degradation product comprises nitrobenzoic acid, the nitrobenzoic acid may comprise/consist essentially/consist of a single isomer such as p-nitrobenzoic acid or 4-nitrobenzoic acid. Advantageously, in such embodiments, as only a single isomer (e.g., 4-nitrobenzoic acid) is obtained as the oxidation/degradation product, this eliminates the need for a post-oxidation step to isolate/separate a desired isomer (e.g., 4-nitrobenzoic acid) from a final product containing a mixture of isomers (e.g., a product mixture containing 2-nitrobenzoic acid, 3-nitrobenzoic acid and/or 4-nitrobenzoic acid isomers).

In various embodiments, the oxidizing/degrading step comprises one or more of the following steps: dispersing, mixing, stirring, sonicating, ultrasonicating and/or heating the mixture comprising:
(a) benzenoid-based polymer source;
(b) NOₓ source;
(c) oxygen-containing gas;
(d) optionally catalyst; and
(e) optionally a green/environmentally friendly/environmentally benign solvent.

In various embodiments, the method further comprises a step of isolating/recrystallizing the oxidation/degradation products. In various embodiments, the isolating/recrystallizing step comprises one or more of the following steps: cooling, drying, purifying, centrifuging, quenching, dissolving, washing, filtering and/or decanting the oxidation/degradation products to remove impurities such as excess benzenoid-based polymer, catalyst and/or unreacted compounds. The step(s) of cooling, drying, purifying, centrifuging, quenching, dissolving, washing, filtering and/or decanting may be repeated at least 1 time, at least 2 times, at least 3 times, at least 4 times, at least 5 times, at least 6 times, at least 7 times, at least 8 times, at least 9 times, or at least 10 times with a recrystallization/washing medium. In various embodiments, the recrystallization solvent or washing medium comprises an aqueous medium such as water.

In various embodiments, the method further comprises, prior to the oxidizing/degrading step, a pre-oxidation step to reduce the original size (and/or increase the surface area) of the benzenoid-based polymer containing source. The pre-oxidation step may comprise a step of breaking/ripping/tearing/cutting the benzenoid-based polymer source into smaller pieces/chunks. It will be appreciated that, in various embodiments, the pre-oxidation step is not a strict requirement of the method although said step increases the rate of conversion/reaction due to the larger exposed surface area (as a result of breaking/ripping/tearing/cutting the benzenoid-based polymer source).

In various embodiments, the step of oxidizing/degrading comprises adding/incorporating from about 0.10 mol % to about 99.9 mol %, from about 0.20 mol % to about 99.8 mol %, from about 0.30 mol % to about 99.7 mol %, from about 0.40 mol % to about 99.6 mol %, from about 0.50 mol % to about 99.5 mol %, from about 1.00 mol % to about 99.0 mol %, from about 2.00 mol % to about 98.0 mol %, from about 3.00 mol % to about 97.0 mol %, from about 4.00 mol % to about 96.0 mol %, from about 5.00 mol % to about 95.0 mol %, from about 10.0 mol % to about 90.0 mol %, from about 15.0 mol % to about 85.0 mol %, from about 20.0 mol % to about 80.0 mol %, from about 25.0 mol % to about 75.0 mol %, from about 30.0 mol % to about 70.0 mol %, from about 35.0 mol % to about 65.0 mol %, from about 40.0 mol % to about 60.0 mol %, from about 45.0 mol % to about 55.0 mol %, or about 50.0 mol % of the benzenoid-based polymer source with respect to/based on the reaction mixture. For example, the reaction mixture may be prepared using about 10 mol % of catalyst (or about 0.1 mmol catalyst), about 0.2 ml NOₓ/nitrate source (or about 3.15 mmol of 70% HNO₃), about 2 mL solvent (or about 35 mmol solvent); and about 1.0 mmol of benzenoid-based polymer source (e.g., polystyrene) per repeating unit.

In various embodiments, the step of oxidizing/degrading comprises adding/incorporating from about 1.00 mol % to about 10.00 mol %, from about 1.50 mol % to about 9.50 mol %, from about 2.00 mol % to about 9.00 mol %, from about 2.50 mol % to about 8.50 mol %, from about 3.00 mol % to about 8.00 mol %, from about 3.50 mol % to about 7.50 mol %, from about 4.00 mol % to about 7.00 mol %, from about 4.50 mol % to about 6.50 mol %, from about 5.00 mol % to about 6.00 mol %, or about 5.50 mol % of the NOₓ source with respect to/based on the reaction mixture.

In various embodiments, the molar ratio of the number of repeating units (e.g., styrene repeating units) present in the benzenoid-based polymer (e.g., polystyrene) to the NOₓ source is from about 1:1 to about 1:10, from about 1:2 to about 1:9, from about 1:3 to about 1:8, from about 1:4 to about 1:7, from about 1:5 to about 1:6, or about 1:5.5. In various embodiments, the step of oxidizing/degrading comprises adding NOₓ source at about 1 molar equivalent, about 2 molar equivalent, about 3 molar equivalent, about 4 molar equivalent, about 5 molar equivalent, about 6 molar equivalent, about 7 molar equivalent, about 8 molar equivalent, about 9 molar equivalent, or about 10 molar equivalent with respect to the number of repeating units present in the benzenoid-based polymer (e.g., value of n in general formula (1)).

In various embodiments, the step of oxidizing/degrading is performed in the presence of from about 0.0 mol % to about 20.0 mol %, from about 1.0 mol % to about 20.0 mol %, from about 1.5 mol % to about 19.5 mol %, from about 2.0 mol % to about 19.0 mol %, from about 2.5 mol % to about 18.5 mol %, from about 3.0 mol % to about 18.0 mol %, from about 3.5 mol % to about 17.5 mol %, from about 4.0 mol % to about 17.0 mol %, from about 4.5 mol % to about 16.5 mol %, from about 5.0 mol % to about 16.0 mol %, from about 5.5 mol % to about 15.5 mol %, from about 6.0 mol % to about 15.0 mol %, from about 6.5 mol % to about 14.5 mol %, from about 7.0 mol % to about 14.0 mol %, from about 7.5 mol % to about 13.5 mol %, from about 8.0 mol % to about 13.0 mol %, from about 8.5 mol % to about 12.5 mol %, from about 9.0 mol % to about 12.0 mol %, from about 9.5 mol % to about 11.5 mol %, from about 10.0 mol % to about 11.0 mol %, or about 10.5 mol % of the catalyst with respect to/based on the reaction mixture. In various embodiments, the efficiency of the method performed with 1.0 mol % of catalyst may be of similar/comparable efficiency as the method performed with 10.0 mol % of catalyst. Accordingly, the catalyst may be present in an amount of about 1 mol % with respect to the reaction mixture to minimise wastage.

In various embodiments, the step of oxidizing/degrading is performed in the presence of from about 0.0 mol % to about 99.9 mol %, from about 50.0 mol % to about 99.9 mol %, from about 0.10 mol % to about 99.9 mol %, from about 0.20 mol % to about 99.8 mol %, from about 0.30 mol % to about 99.7 mol %, from about 0.40 mol % to about 99.6 mol %, from about 0.50 mol % to about 99.5 mol %, from about 1.00 mol % to about 99.0 mol %, from about 2.00 mol % to about 98.0 mol %, from about 3.00 mol % to about 97.0 mol %, from about 4.00 mol % to about 96.0 mol %, from about 5.00 mol % to about 95.0 mol %, from about 10.0 mol % to about 90.0 mol %, from about 15.0 mol % to about 85.0 mol %, from about 20.0 mol % to about 80.0 mol %, from about 25.0 mol % to about 75.0 mol %, from about 30.0 mol % to about 70.0 mol %, from about 35.0 mol % to about 65.0 mol %, from about 40.0 mol % to about 60.0 mol %, from about 45.0 mol % to about 55.0 mol %, or about 50.0 mol % of the solvent with respect to/based on the reaction mixture. In various embodiments, the NOₓ source is mixed with the solvent in a molar ratio of from about 1:1 to about 1:20, from about 1:2 to about 1:19, from about 1:3 to about 1:18, from about 1:4 to about 1:17, from about 1:5 to about 1:16, from about 1:6 to about 1:15, from about 1:7 to about 1:14, from about 1:8 to about 1:13, from about 1:9 to about 1:12, from about 1:10 to about 1:11, or about 1:10.5. In various embodiments, the solvent is present in an amount of about 2 mL per 100 mg of benzenoid-based polymer (e.g., polystyrene).

In various embodiments, the method achieves a conversion (of the total amount/number of oxidation/degradation products) of at least about 5.0%, at least about 10.0%, at least about 15.0%, at least about 20.0%, at least about 25.0%, at least about 30.0%, at least about 35.0%, at least about 40.0%, at least about 45.0%, at least about 50.0%, at least about 55.0%, at least about 60.0%, at least about 65.0%, at least about 70.0%, at least about 75.0%, at least about 80.0%, at least about 81.0%, at least about 82.0%, at least about 83.0%, at least about 84.0%, at least about 85.0%, at least about 86.0%, at least about 87.0%, at least about 88.0%, at least about 89.0%, at least about 90.0%, at least about 91.0%, at least about 92.0%, at least about 93.0%, at least about 94.0%, at least about 95.0%, at least about 96.0%, at least about 97.0%, at least about 98.0%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9%.

In various embodiments, the method achieves a yield (of the total amount/number of oxidation/degradation products) of at least about 5.0%, at least about 10.0%, at least about 15.0%, at least about 20.0%, at least about 25.0%, at least about 30.0%, at least about 35.0%, at least about 40.0%, at least about 45.0%, at least about 50.0%, at least about 55.0%, at least about 60.0%, at least about 65.0%, at least about 67.0%, at least about 70.0%, at least about 75.0%, at least about 80.0%, at least about 81.0%, at least about 82.0%, at least about 83.0%, at least about 84.0%, at least about 85.0%, at least about 86.0%, at least about 87.0%, at least about 88.0%, at least about 89.0%, at least about 90.0%, at least about 91.0%, at least about 92.0%, at least about 93.0%, at least about 94.0%, at least about 95.0%, at least about 96.0%, at least about 97.0%, at least about 98.0%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9%. In various embodiments, the yield increases/improves with an increase in one or more of the following: temperature, pressure and/or time duration used/applied/employed during the oxidizing/degrading step.

In various embodiments, the method achieves a yield (of benzoic acid) of at least about 5.0%, at least about 10.0%, at least about 15.0%, at least about 20.0%, at least about 25.0%, at least about 30.0%, at least about 35.0%, at least about 40.0%, at least about 45.0%, at least about 50.0%, at least about 52.0%, at least about 55.0%, at least about 60.0%, at least about 65.0%, at least about 70.0%, at least about 75.0%, at least about 80.0%, at least about 81.0%, at least about 82.0%, at least about 83.0%, at least about 84.0%, at least about 85.0%, at least about 86.0%, at least about 87.0%, at least about 88.0%, at least about 89.0%, at least about 90.0%, at least about 91.0%, at least about 92.0%, at least about 93.0%, at least about 94.0%, at least about 95.0%, at least about 96.0%, at least about 97.0%, at least about 98.0%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9%. In various embodiments, the yield increases/improves with an increase in one or more of the following: temperature, pressure, time duration and/or amount of NOₓ source (e.g., HNO₃) used/applied/employed during the oxidizing/degrading step.

In various embodiments, the method achieves a yield (of 4-nitrobenzoic acid) of at least about 5.0%, at least about 10.0%, at least about 15.0%, at least about 20.0%, at least about 25.0%, at least about 30.0%, at least about 35.0%, at least about 40.0%, at least about 45.0%, at least about 50.0%, at least about 55.0%, at least about 60.0%, at least about 65.0%, at least about 70.0%, at least about 75.0%, at least about 80.0%, at least about 81.0%, at least about 82.0%, at least about 83.0%, at least about 84.0%, at least about 85.0%, at least about 86.0%, at least about 87.0%, at least about 88.0%, at least about 89.0%, at least about 90.0%, at least about 91.0%, at least about 92.0%, at least about 93.0%, at least about 94.0%, at least about 95.0%, at least about 96.0%, at least about 97.0%, at least about 98.0%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9%. In various embodiments, the yield increases/improves with an increase in one or more of the following: temperature, pressure, time duration and/or amount of NOₓ source (e.g., HNO₃) used/applied/employed during the oxidizing/degrading step.

In various embodiments, the method is performed in a fume hood, e.g., in a well-ventilated fume hood. In various embodiments, performing the method in a fume hood aids in ease of removal of gaseous intermediates (e.g., excess NOₓ such as NO, NO₂ and O₂) that are produced during the reaction. Advantageously, carrying out the oxidation reaction in a fume hood avoids the possibility of a pressure build-up of gaseous intermediates (e.g., excess NOₓ such as NO, NO₂ and O₂) that are produced during the reaction, which would otherwise occur if the reaction were to be carried out in an enclosed pressurized system.

Advantageously, in various embodiments, carrying out the oxidation reaction in a fume hood also provides more safety and protects the user from hazardous materials by reducing/eliminating exposure to hazards such as toxic fumes from chemicals and solvents, chemical spills/splashes etc.

In various embodiments, the method is performed in glass reactors and/or reactors containing an acid-resistant protective layer/coating. Advantageously, carrying out the reaction in such reactors avoids the possibility of corrosion by the use of acetic acid and nitrate sources such as nitric acid, which would otherwise occur if the reaction were to be carried out in metal vessels.

In various embodiments, the method does not require and/or is substantially devoid of the use of expensive and/or complex/sophisticated equipment that requires high energy input. Advantageously, in various embodiments, the method utilizes a simple and straightforward experimental set up. In various embodiments, the method is cost effective (i.e. has a low production cost). Advantageously, embodiments of the method disclosed herein have a high production yield, high scalability and/or high versatility (e.g., can be used for a wide range of benzenoid-based polymer waste).

In various embodiments, there is provided a material comprising the oxidation/degradation products as disclosed herein. In various embodiments, the material is suitable for use in applications such as perfume, dye, dye intermediate, topical medication, insect repellent, antimicrobial application, intermediate/precursor in manufacture of chemicals (e.g., folic acid), preservative, antibacterial agent and/or fuel cell.

In various embodiments, the method disclosed herein allows benzenoid-based polymer containing source/waste to be converted/upcycled/recycled into oxidation products through the synergistic combination of the use of a NOₓ source, an oxygen-containing gas and at least one of an environmentally benign solvent or a catalyst. Advantageously, embodiments of the method disclosed herein allow for simple, cheap, cost-efficient, energy-efficient (e.g., less energy intensive) and sustainable conversion/transformation of benzenoid-based polymer containing source/waste into useful and simpler chemicals. Embodiments of the method efficiently upcycle/recycle/recover chemicals from benzenoid-based polymer containing source/waste, without the need for expensive and/or tedious/complex/sophisticated chemical upcycling/recycling/recovering techniques that require high energy input (e.g., pyrolysis etc) or light (e.g., photoreactions). In various embodiments, the method disclosed herein is scalable for industrial applications.

Advantageously, embodiments of the method are less energy intensive as compared to currently reported thermal degradation reactions; use a green solvent and metal-free catalyst that are commercially available or can be synthesized in one step under green conditions; minimally affected by additives such as dyes commonly present in plastic waste; easy to perform as it is carried out in air under atmospheric pressure; and/or provides/achieves comparable yields to currently reported literatures within similar or shorter reaction time

In various embodiments, the benzenoid-based polymer comprises polystyrene. The polystyrene may be derived from a polystyrene containing source which includes but is not limited to polystyrene-based waste, polystyrene waste, waste polystyrene, unwanted polystyrene or the like. In various embodiments, the polystyrene containing source comprises waste polystyrene. In various embodiments, the method comprises recycling/upcycling waste polystyrene into valuable products/chemicals. Advantageously, in various embodiments, the method disclosed herein allows waste polystyrene to be recycled/upcycled into forms that are useful on their own or useful as feedstocks for further applications. In various embodiments, the material is derived from benzenoid-based polymer source/waste (e.g., polystyrene source/waste).

Embodiments of the present technology are different from those of the art that focuses on the oxidation of small molecules such as 2,4,6-trinitrotoluene (TNT). As such small molecules are soluble in most organic solvents, the same cannot be said for benzenoid-based polymers. For instance, it would have been entirely unexpected that green reagents such as acetic acid and/or nitric acid could be used when working with benzenoid-based polymers (e.g., polystyrene) that are known to have low solubility in acetic acid. It will be appreciated that benzenoid-based polymers are only soluble in limited number of solvents such as chlorinated solvents or aromatic solvents (and some common solvents such as tetrahydrofuran (THF) and acetone). Additionally, the solvents that can be used to contain/dissolve benzenoid-based polymer such as polystyrene (i.e. chlorinated and/or aromatic solvents) are not compatible with nitric acid under heating. Furthermore, in contrast to performing oxidation on small molecules (such as toluenes), the oxidative cleavage of benzenoid-based polymer such as polystyrene (PS) (e.g., to benzoic acids) is mechanistically far more complex and involves many elementary reaction steps e.g., H-abstraction, beta-scission, O₂ combinations etc. Accordingly, there is simply no reason to expect that the reaction conditions/reactants/reagents used for oxidation of small molecules could be suitably used for oxidation of benzenoid-based polymers. On the contrary, the present application has surprisingly found that embodiments of the present technology comprising the synergistic combination of common reagents (e.g. acetic acid and nitric acid along with THICA) could successfully convert aromatic polymer (e.g., polystyrene) into oxidation products, which are not shown and cannot be expected from the art.

Embodiments of the present technology are different from those of the art that uses pure O₂ at a relatively high pressure of about 2 atmospheric pressure (0.2 MPa). In contrast, embodiments of the present method is able to work with air (containing 21% O₂) at 1 atmospheric pressure. Advantageously, despite the lower concentration of O₂ used in embodiments of the presently disclosed method as compared to those in the art, the present application has shown that performing oxidation on polymers comprising benzenoid rings (e.g., polystyrene) according to embodiments of the present technology give appreciable yields of valuable chemicals such as benzoic acids.

Embodiments of the present technology are different from those of the art that uses only NHPI as the catalyst. It will be appreciated that using NHPI alone is not a sufficiently strong catalyst for the degradation of polymers into smaller molecules, even in the presence of the highly-oxidising combination of NaClO and NaBr. Indeed, using only NHPI as the catalyst results in a partial oxidation of a polymer, such as cellulose, where the polymers were not significantly depolymerized and only the side chain hydroxyl groups were oxidized. On the contrary, the present application has shown that embodiments of the present technology comprising the reactive synergistic combination of using NHPI-derivatives, nitrate sources, and an O₂-containing atmosphere in acetic acid could successfully convert aromatic polymer (e.g., polystyrene) into oxidation products with high yield, which are not shown and cannot be expected from the art.

Embodiments of the present technology are different from those of the art that focuses on the oxidation of small molecules, by using polystyrene resin as an inert support in which THICA was chemically fixed onto.

Embodiments of the present technology are different from those of the art in that embodiments of the present technology comprise the use of a nitrate source. Particularly, it was found that the nitrous oxides produced are the reactive intermediates that further cleaves the oxidized polystyrene and breaks it down into smaller molecules such as benzoic acids.

Embodiments of the present technology are different from those of the art that use a more traditional NHPI with Co/Mn metal oxidation conditions, which is well-known for enhancing the rates of hydrocarbon oxidation. Using such methods for performing oxidation of polystyrene results in an inefficient reaction, and reasonable product yields can only be obtained at high temperatures (205° C.) and very high pressures (1000 psi=68 bars).

Embodiments of the present technology are different from those of the art that focus on only oxidation of small molecules. These small molecules have properties that are starkly different from a polymeric molecule like polystyrene and there is therefore simply no reason to expect that the reaction conditions used for oxidation of small molecules could be suitably used or replicated for oxidation of benzenoid-based polymers. Furthermore, oxidative degradation of polymers such as PS is mechanistically more complicated than the oxidation of small molecule hydrocarbons, and therefore the success of the latter cannot be translated to facile polymer oxidation. Embodiments of the present technology are different from those of the art that use a more traditional way of oxidation, that is, using Co or Mn salts as an oxidizer rather than nitrates. In contrast to embodiments of the present technology, using such metal salts in combination with THICA does not work efficiently on polystyrene.

Embodiments of the present technology are completely different from those of the art that perform oxidative degradation of different plastics (including polystyrene) into smaller molecules by using oxygen (O₂) and nitrogen oxide (NO) gas as the only reagent without using any solvents, under highly hazardous reaction conditions that are high in energy demand (i.e. at a high temperature of 160° C. and high pressure ranging from 275 kPa to 3170 kPa.

Embodiments of the present technology are completely different from those of the art that performs photo-oxidation at room temperature using catalytic amounts of FeCl₃, tert-butyl ammonium chloride and 2,2,2-trichloroethanol, with O₂ as the terminal oxidant.

Embodiments of the present technology are completely different from those of the art that performs photo-oxidation at room temperature using a catalytic amount of FeCl₃, with O₂ as the terminal oxidant. It will be appreciated that such a method of the art is not applicable for use on actual waste plastic samples as dyes present in actual waste plastic samples would result in lower efficiency because of the inhibition of light penetration.

Embodiments of the present technology are completely different from those of the art that performs photo-oxidation at room temperature using different acid catalysts, with O₂ as the terminal oxidant and benzene as a highly toxic solvent. It will be appreciated that such a method of the art is not applicable for use on actual waste plastic samples as dyes present in actual waste plastic samples would result in lower efficiency because of the inhibition of light penetration.

Embodiments of the present technology is completely different from those of the art that require harsh working conditions (i.e. 190° C., 20 atm with an oxidative gas mixture), heavy metals containing catalyst (Co/Mn/Br), toxic aromatic solvent (i.e. benzene), and operate by purging with N₂ and constant feedstock flow of O₂.

Embodiments of the present technology is completely different from those of the art that require harsh working conditions (i.e. 220° C., 68 atm), heavy metals containing catalyst (Co/Mn/Br/Zn), toxic aromatic or chlorinated solvent (i.e. benzene, chlorobenzene and o-dichlorobenzene), and operate in O₂ gas under high pressure.

In summary, it is believed that various embodiments of the present technology are not easily arrived at and are advantageous over known methods for one or more of the reasons discussed below:
The properties of polymers such as polystyrene differ from small molecules such as ethylbenzene (monomer unit of polystyrene). One such case is solubility. Although NHPI derivatives are known catalysts for radical oxidations, such catalysts have only been used effectively for oxidising small molecules. As noted above, the oxidative degradation of hydrocarbon polymers is mechanistically far more complicated than simply oxidizing small molecules, as this involves a number of elementary mechanistic steps (e.g. beta-scission) that are not required for the latter. Therefore, the ability of reaction conditions to oxidise small molecules do not necessarily translate to a similar capability for polymers. For example, in some of the prior art methods where the reported conditions effectively oxidise small molecules, they are inert to the polystyrene resin support used in the reactions.
For polymers such as polystyrenes, NHPI alone as a catalyst are normally ineffective or minimally effective in methods of the art where the use of NHPI and Co metal in the oxidation of polystyrene only gave low yield (e.g. less than 15%) even at high temperature and pressure of 180° C. and 1000 Psi.
During experimental trials, it is also surprisingly discovered that combination of the different reaction components disclosed can degrade the polymer more efficiently than if only one of the reaction components are used.
As compared to small molecules, plastic polymers such as polyethylene are notorious for their poor solubilities in many common organic solvents. Although polystyrene is slightly better, the available choice of solvent is limited to harmful chlorinated or aromatic solvents. Hence, many reports of polystyrene degradation are done in such solvents or neat reactions under harsh conditions and conditions that was used on small molecules cannot be easily replicated onto a polymer with good efficiency even if the polymer in theory may have similar reactivity. Furthermore, it has already been reported that traditional oxidation using NHPI with metal Co/Mn on polystyrene gave low yields even under harsh conditions. This would have deterred one from trying the degradation with the same/similar conditions/reactants again.
It is known that nitrate salts have poor solubility in chlorinated solvents which are used in prior art methods, hence nitrate salts would not have been a preferred choice by those skilled in the art since it is expected not to be efficient enough. Using nitric acid is also not sufficiently motivated due to its incompatibility with most organic solvents. Additionally, acetic acid is known to not be able to dissolve polystyrene and thus there is little or no motivation to consider such solvent for use. Hence, it is not obvious and indeed counter intuitive for a person skilled in the art to arrive at embodiments of the presently disclosed method of degradation that use the combination of these reagents.
Experiments carried out have shown that the product yield may be even further enhanced by staggering time of nitrate source addition, which is nowhere taught before.
Other than benzoic acid formation (which most polystyrene degradation reaction aims to form), various embodiments disclosed herein also formed p-nitrobenzoic acid as the secondary product. This was the by-product formed from using a nitrate source. Notably, in various embodiments, p-nitrobenzoic acid may be formed exclusively and not any of the other isomers. This product has many uses including being a precursor to folic acid, a common pregnancy drug. It can be also separated easily from benzoic acid but not as easy from its isomers. Hence, the ability to form p-nitrobenzoic acid exclusively is indeed a technical advantage as other ways to form this compound have shown the presence of its isomers in the mix.
Embodiments of the present disclosure are also versatile enough to be used on different types of polystyrene substrates—embodiments of the disclosed method could form benzoic acids from products containing different dyes and additives. As these compounds (especially dyes) contain easily-oxidisable unsaturated functional groups (e.g. C═C, OH) that can be oxidised at the expense of PS, this demonstrates the practicality of the embodiments of presently disclosed process as these compounds do not appear to significantly hinder embodiments of the currently disclosed reaction. This technical effect is not obvious at all from the methods in the art and cannot be predicted a priori. This is also an obvious advantage over other methods of light-driven PS oxidation which do not work well with strongly-coloured substrates.
The use of acetic acid in embodiments of the degradation method is not obvious as polystyrene does not dissolve in acetic acid. The reaction sped up after the initial cleaving of polystyrene into smaller chains that is more soluble and finally into the final benzoic acids. In embodiments of the disclosed method, styrofoam boxes may be broken into chunks and added to the reaction without first powdering them (to increase the rate of reaction). Hence, the practicality is enhanced as no pre-processing of PS needs to take place.

### BRIEF DESCRIPTION OF FIGURES

FIG. 1 is a schematic diagram 100 showing a method of converting a polymer comprising benzenoid rings or a benzenoid-based polymer (derived from a benzenoid-based polymer source) into oxidation/degradation product(s) in accordance with various embodiments.

FIG. 2 is a schematic diagram 200 showing a method of converting waste polystyrene into oxidation/degradation product(s) in accordance with various embodiments disclosed herein.

FIG. 3 shows photographs of five different types of polystyrene (PS) products used during the experiments described in Example 3.

FIG. 4 shows ¹H NMR spectrum of polystyrene (PS) beads (140 kDa) in CDCl₃ in accordance with various embodiments disclosed herein.

FIG. 5 shows ¹H NMR spectrum of degraded polystyrene (PS) beads (140 kDa) mixture in CDCl₃ in accordance with various embodiments disclosed herein. 1,2-dichloroethane (DCE) was added as an internal standard. Signal a corresponds to protons on p-nitrobenzoic acid. Signals b, c and d correspond to protons on benzoic acid.

FIG. 6 shows ¹H NMR spectra of various waste polystyrene (PS) products stacked together with the ¹H NMR spectra of their respective degraded mixtures in CDCl₃ in accordance with various embodiments disclosed herein. The range of 6.00-8.60 ppm is shown for the ¹H NMR spectra.

FIG. 7 shows ¹H NMR spectrum of pure benzoic acid/p-nitrobenzoic acid (BA/NBA) products obtained after 1.04 g of styrofoam degradation (after 24 hours and after recrystallization) in CDCl₃ in accordance with various embodiments disclosed herein.

FIG. 8 shows ¹H NMR spectrum of the pure benzoic acid/p-nitrobenzoic acid (BA/NBA) products obtained from Example 4 (as shown in FIG. 7) after 1.04 g of styrofoam degradation in CDCl₃ in accordance with various embodiments disclosed herein, stacked together with ¹H NMR spectrum of commercial benzoic acid and ¹H NMR spectrum of commercial p-nitrobenzoic acid in CDCl₃. Signal a corresponds to protons on p-nitrobenzoic acid. Signals b, c and d correspond to protons on benzoic acid.

FIG. 9 shows ¹H NMR spectrum of a crude reaction mixture in CDCl₃ in accordance with various embodiments disclosed herein, stacked together with 1H NMR spectrum of commercial benzoic acid and ¹H NMR spectrum of commercial p-nitrobenzoic acid. Signal a corresponds to protons on p-nitrobenzoic acid. Signals b, c and d correspond to protons on benzoic acid. Signal e corresponds to protons on formic acid.

### EXAMPLES

Example embodiments of the disclosure will be better understood and readily apparent to one of ordinary skill in the art from the following examples, tables and if applicable, in conjunction with the figures. It should be appreciated that other modifications related to structural, and/or chemical changes may be made without deviating from the scope of the invention. Example embodiments are not necessarily mutually exclusive as some may be combined with one or more embodiments to form new example embodiments. The example embodiments should not be construed as limiting the scope of the disclosure.

In the following examples, a metal-free and environmentally benign/sustainable methodology has been developed for converting waste polymer comprising benzenoid rings (e.g., waste benzenoid-based polymer source) into useful smaller molecules such as benzoic acid, p-nitrobenzoic acid and formic acid. The approach of the examples comprises use of a thermal-based (e.g., thermal oxidative) degradation under a reasonably low temperature and atmospheric pressure, with the use of green solvents and air as terminal oxidant. In addition, the catalysts used during the thermal-based degradation are commercially-available or can be synthesized easily under environmentally-benign conditions, while the nitrate sources used during the thermal-based degradation are cheap and readily-available.

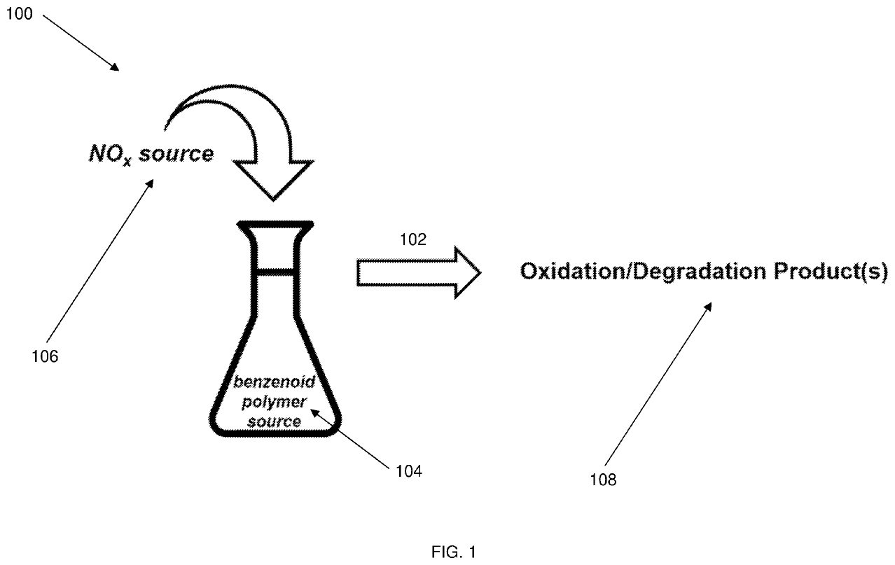

A method of converting a polymer comprising benzenoid rings (or a benzenoid-based polymer source) into oxidation/degradation product(s) in accordance with various embodiments disclosed herein is shown in FIG. 1. In step 102, a benzenoid-based polymer source 104 is oxidized/degraded by adding a NOₓ source 106 to the benzenoid-based polymer source 104 under suitable conditions, thereby obtaining oxidation/degradation product(s) 108.

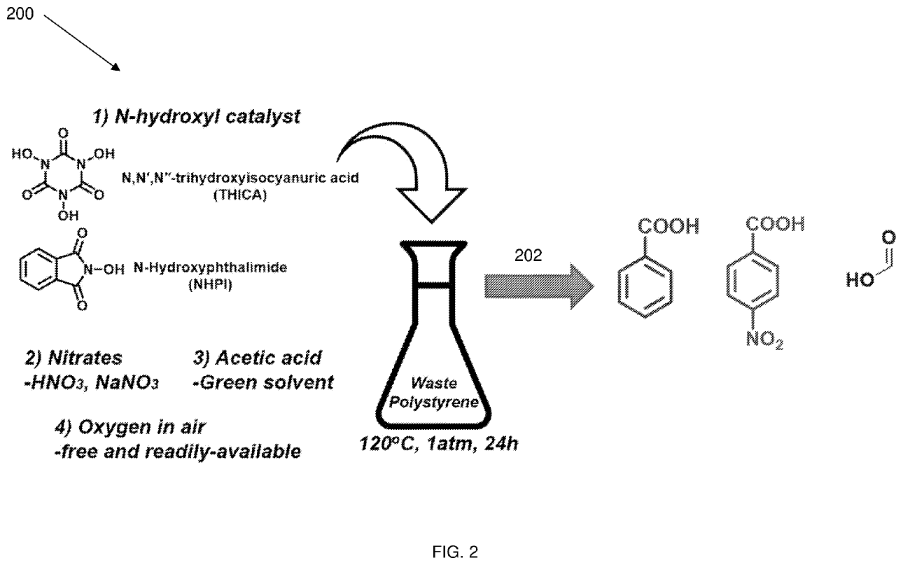

In the following examples 1 to 7, polystyrene is used as an example of a benzenoid-based polymer source. A method of converting waste polystyrene into oxidation/degradation product(s) in accordance with various embodiments disclosed herein is shown in FIG. 2. In step 202, waste polystyrene is oxidized/degraded in the presence of (1) N-hydroxyl catalyst (e.g., THICA and/or NHPI); (2) nitrates (e.g., HNO₃ and/or NANO₃); (3) acetic acid; and (4) air to obtain oxidation/degradation product(s). The oxidation/degradation products may be benzoic acid, p-nitrobenzoic acid and/or formic acid. Step 202 may be carried out with heating at a temperature of about 120° C. and pressure at about 1 atm (ambient pressure conditions) over a duration of 24 hours.

The following examples describe a thermal oxidative degradation of polystyrene under mild, metal-free conditions within 24 hours with organocatalysts containing N-hydroxyl groups as the key catalyst and a nitrate source such as HNO₃. Advantageously, the resulting product is a mixture of useful chemicals like benzoic acid, p-nitrobenzoic acid and formic acid. Benzoic acid has various applications in the production of perfumes, dyes, topical medications, insect repellents and antimicrobial applications while p-nitrobenzoic acid can be used as an intermediate and precursor in the manufacture of folic acid, p-aminobenzoic acid, and dye intermediate. Formic acid has various applications in the production of preservative, antibacterial agent, hydrogen carrier and fuels for formic acid cells.

As will be shown in the examples, the inventors have demonstrated that it is possible to perform organocatalytic (metal-free) thermal-based oxidative degradation of polystyrene into aromatic acids, benzoic acid and/or p-nitrobenzoic acid under mild conditions (e.g., 120° C. and/or 100 kPa), that is minimally-affected by the presence of additives and dyes in an industrially-relevant process. Air (1 bar) was used as a cheaper alternative instead of a pure oxygen atmosphere. The inventors also demonstrated that it is possible to convert polystyrene into aromatic acids, benzoic acid and/or p-nitrobenzoic acid in an environmentally-sustainable method by using just benign acetic acid (used in household vinegar) without any chlorinated or aromatic solvents, as well as not using any metals. The possibility of using acetic acid (i.e. a green solvent) for a thermal-based oxidative degradation of polystyrene may not be easily envisaged since it is not expected for degradation of polystyrene to be successfully performed in solvents other than aromatic/chlorinated solvents. Furthermore, the method is capable of degrading different types of polystyrene products regardless of dyes or additives. To the best of the inventors' knowledge, such a method of converting polystyrene into aromatic acids using organocatalysis under environmentally-sustainable thermal conditions has not been previously reported before. In addition, the usage of non-metal catalyst (e.g., N-hydroxyl catalysts such as THICA) on polystyrene oxidative cleavage, and nitrate sources has also not been reported previously and cannot be predicted without any trials or easily envisaged.

Advantageously, a method has been developed that allows waste polymer comprising benzenoid rings (e.g., waste benzenoid-based polymer source) to be upcycled in a less energy intensive (and therefore more cost and energy efficient) manner and under environmentally-sustainable conditions. Advantageously, the method is less energy intensive (e.g., 120° C.) than current pyrolysis methods. The method/reaction is also easy to perform as it is carried out in air under atmospheric pressure. Advantageously, the method uses a green solvent (e.g., acetic acid) and metal-free catalyst which is commercially available or can be synthesized from commercial precursors in one step under green conditions, thereby making the method an environmentally friendly and sustainable one. Advantageously, the method is able to degrade different types of polystyrene products and shows resilience to dyes or additives.

### Example 1: Oxidative Degradation of Polystyrene

A thermal oxidative degradation of PS into BA and NBA using N-hydroxyphthalimide (NHPI) catalyst and its derivatives such as THICA has been developed. Instead of using gaseous reactants such as NOₓ (g), common nitrates such as HNO₃ and NaNO₃ were used as a NOₓ source via thermal decomposition. Using THICA (Table 1, Entry 1) as a catalyst in the presence of HNO₃ as the oxidant and AcOH as the solvent under O₂, a combined yield of 27% was obtained for BA and NBA after 24 hours. The reaction works efficiently as well when conducted under atmospheric conditions (Table 1, Entry 5).

Next, experiments were conducted where two other commercially available catalysts, NHPI and N-hydroxytetrachlorophthalimide (Cl₄NHPI) were used in place of THICA. It was shown that the yield obtained when using NHPI was comparable to the yield obtained when using THICA (Table 1, Entry 3) while Cl₄—NHPI was less efficient (Table 1, Entry 4).

As thermal decomposition of HNO₃ can occur starting from 80° C., the experiments were conducted with two other temperatures, namely 80° C. and 120° C., to determine the effects of the change in temperature (Table 1, Entry 6 and Entry 7). It was found that setting the reaction at 120° C. increased the total yield of the reaction by around 60% (27% at 100° C. vs. 42% at 120° C.). A reaction was performed without the use of N-hydroxyl containing organocatalyst and it was found that the overall yield decreases to 14% from 42% (Table 1, Entry 10 and Entry 7). After a series of condition screenings, the optimal condition required was determined (Table 1, Entry 7).

Other than Entry 7, different conditions can be applied to adjust the ratio of BA and NBA obtained. For instance, using pure HNO₃ (Table 1, Entry 9) gave purely NBA at 17% without any trace of BA. Other than HNO₃, different sources of nitrates such as NaNO₃ can also work in presence of acids such as HCl (Table 1, Entry 8), giving different degree of efficiency and different yields/amounts of BA and NBA.

TABLE 1
Screening of optimized conditions¹

| | | | | | | | | Yield | Yield |
|---|---|---|---|---|---|---|---|---|---|
| | | | Nitrate | | Temperature | Time | Atmosphere | of BA | of NBA |
| Entry | Catalyst | Additive | Source² | Solvent | (° C.) | (hr) | (1 bar) | (%) | (%) |
| 1 | THICA | NA | HNO₃ | AcOH | 100 | 24 | O₂ | 22 | 5 |
| | (10 mol %) | | (0.2 mL) | (2 mL) | | | | | |
| 2 | THICA | CAN | HNO₃ | AcOH | 100 | 24 | Air | 19 | 7 |
| | (10 mol %) | (5 mol %) | (0.2 mL) | (2 mL) | | | | | |
| 3 | NHPI | NA | HNO₃ | AcOH | 100 | 24 | Air | 16 | 6 |
| | (10 mol %) | | (0.2 mL) | (2 mL) | | | | | |
| 4 | Cl₄NHPI | NA | HNO₃ | AcOH | 100 | 24 | Air | 13 | 3 |
| | (10 mol %) | | (0.2 mL) | (2 mL) | | | | | |
| 5 | THICA | NA | HNO₃ | AcOH | 100 | 24 | Air | 20 | 5 |
| | (10 mol %) | | (0.2 mL) | (2 mL) | | | | | |
| 6 | THICA | NA | HNO₃ | AcOH | 80 | 24 | Air | 7 | NR |
| | (10 mol %) | | (0.2 mL) | (2 mL) | | | | | |
| 7 | THICA | NA | HNO₃ | AcOH | 120 | 24 | Air | 32 | 10 |
| | (10 mol %) | | (0.2 mL) | (2 mL) | | | | | |
| 8 | THICA | HCl | NaNO₃ | AcOH | 120 | 24 | Air | 16 | 4 |
| | (10 mol %) | (0.2 mL) | (300 mol %) | (2 mL) | | | | | |
| 9 | THICA | NA | HNO₃ | NA | 120 | 24 | Air | NR | 17 |
| | (10 mol %) | | (2 mL) | | | | | | |
| 10 | NA | NA | HNO₃ | AcOH | 120 | 24 | Air | 10 | 4 |
| | | | (0.2 mL) | (2 mL) | | | | | |

¹Styrofoam was used as the PS source.
²Nitrate source used = 3 mol equivalents with respect to styrene repeating units in PS.
NR = no observable product.
Yields were determined via ¹H NMR with 1,2-dichloroethane (DCE) as the internal standard.

### Example 2: Reaction Procedure

An exemplary reaction procedure employed is described as follows.

104 mg of PS product (1 mmol as per repeating unit) and 18 mg of THICA (0.1 mmol) were added to a 10 mL Schleck tube. 2 mL of acetic acid and 0.2 mL of HNO₃ (~70%) were added. The mixture was heated to 120° C. with stirring under air for 24 hours. After cooling down to room temperature, the reaction mixture was dried under reduced pressure. The solid residue was collected, and the yield of BA and NBA was determined via ¹H NMR using DCE as the internal standard.

### Example 3: Efficiency of Degradation on Different Polystyrene Products

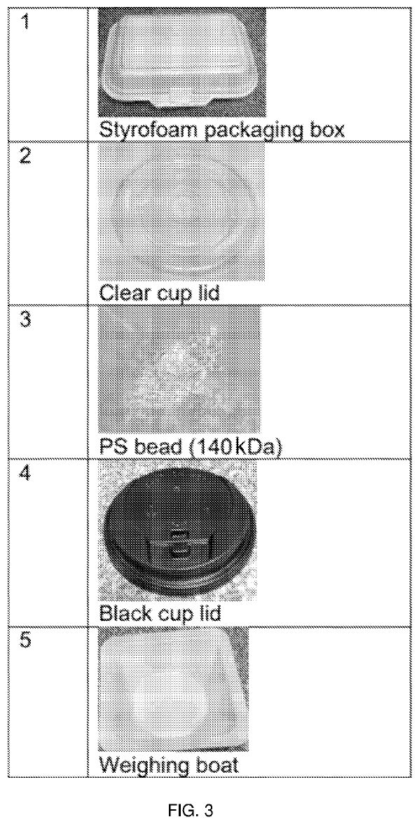

Experiments were conducted on five different types of polystyrene products (FIG. 3) following the reaction procedure described in Example 2 and conditions optimized according to Table 1, Entry 7. The results obtained are shown in Table 2.

TABLE 2
Efficiency of degradation on different polystyrene (PS) products

| | | Yield | Yield of | Total |
|---|---|---|---|---|
| | Type of | of BA | NBA | yield |
| Entry | Polystyrene (PS) | (%) | (%) | (%) |
| 1 | Styrofoam packaging box | 32 | 10 | 42 |
| 2 | Clear cup lid | 30 | 9 | 39 |
| 3 | Polystyrene bead (140 kDa) | 29 | 8 | 37 |
| 4 | Black cup lid | 24 | 7 | 31 |
| 5 | Weighing boat | 21 | 6 | 27 |

*Yield was calculated based on ¹H NMR using DCE as an internal standard.

### Example 4: Product Characterization

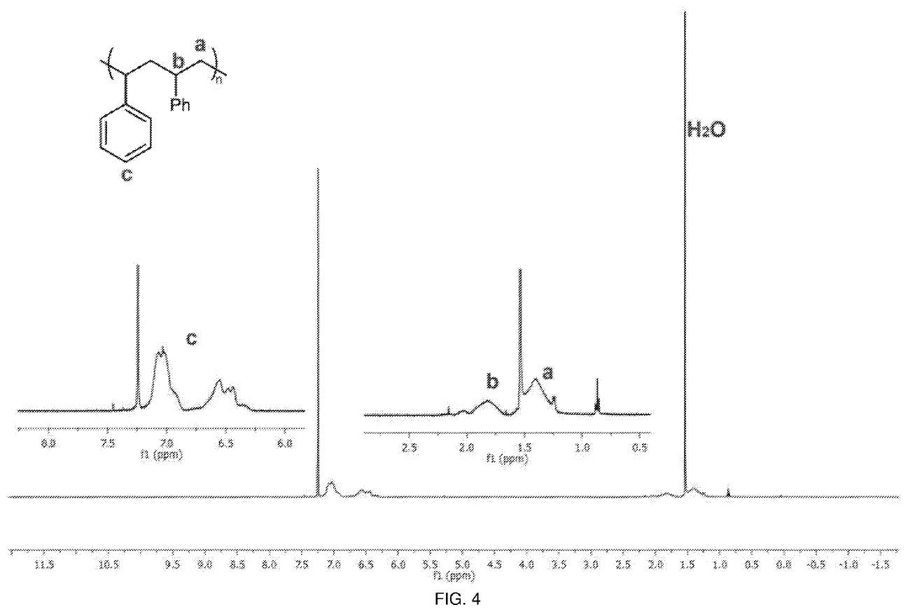

Compositions of the product mixture were analyzed via ¹H NMR and yields were determined using DCE as the internal standards. For PS products in CDCl₃, broad peaks at δ (ppm): 1.15-2.10 depicts the protons on the aliphatic backbone while broad peaks at δ (ppm): 6.30-7.20 depicts the protons on the aromatic rings. The full spectrum for PS beads (140 kDa) is shown as an example in FIG. 4.

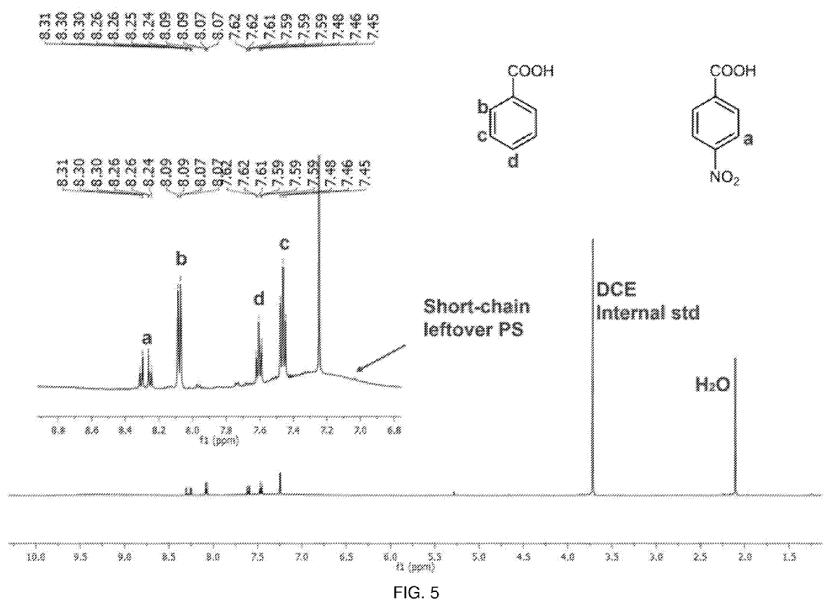

For all the product mixture in CDCl₃, the spectrums generally contain a mixture of BA with peaks at δ (ppm): 7.45-7.48 (t), 7.59-7.62 (t), 8.07-8.09 (d) and NBA with peaks at δ (ppm): 8.24-8.31 (dd). Broad peaks at δ (ppm): 6.80-7.80 depict short chain leftover PS. The full spectrum for degraded PS beads (140 kDa) mixture is shown as an example in FIG. 5.

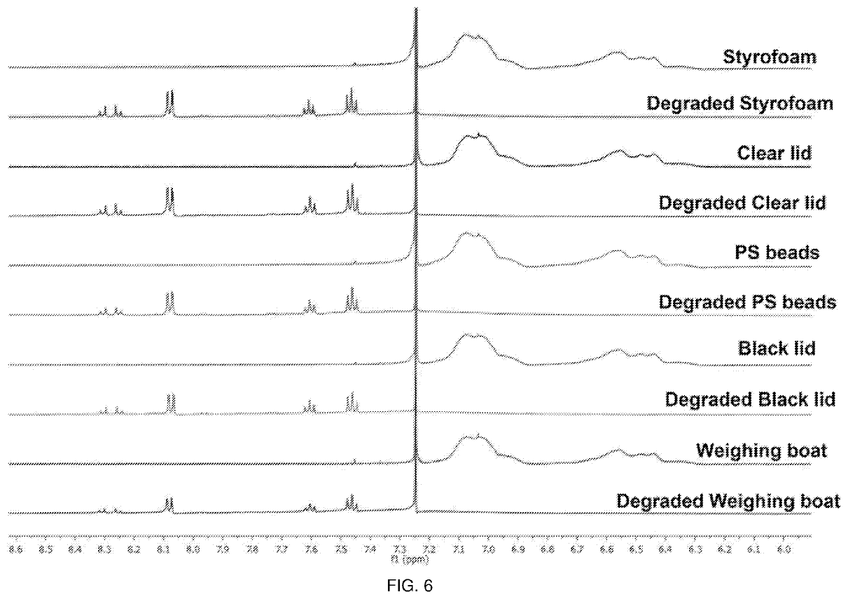

A comparison of all the ¹H NMR spectrums of the PS products along with their degraded mixtures is shown in FIG. 6. Based on the results obtained, it is noticed that the reaction is resilient to additives or dyes that may be present in the PS products (e.g., black dye from black cup lids or plasticizers from weighing boats).

### Example 5: Scale-Up Reaction

The optimized conditions were subjected to a 1.04 g scale reaction using Styrofoam as the PS product. The final product mixture can be purified by simple hot water recrystallization.

1.04 g of Styrofoam (10 mmol as per repeating unit) and 177 mg of THICA (1 mmol) were added to a 100 ml Schleck tube. 20 ml of acetic acid and 2 mL of HNO₃ (~70%) were added. The mixture was heated to 120° C. with stirring under air for 24 hours. After cooling down to room temperature, the reaction mixture was dried under reduced pressure. 5 mL of boiling water was added to the solid residue and stirred. The aqueous mixture was decanted into a 20 mL vial and the process was repeated two more times on the residue solid to obtain three vials of aqueous solution. The aqueous solutions were cooled in the fridge at 0° C. for 1 hour where solids were observed to form. The solids were collected by filtration to obtain a pale yellow crystalline solid. The isolated yield of BA and NBA was determined to be 21% and 5% respectively (via the ratio of BA:NBA by ¹H NMR integration).

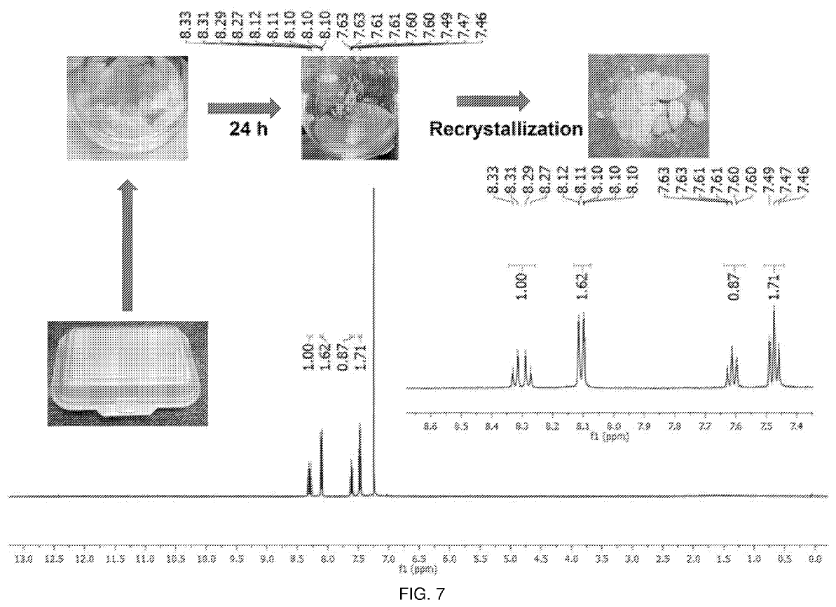

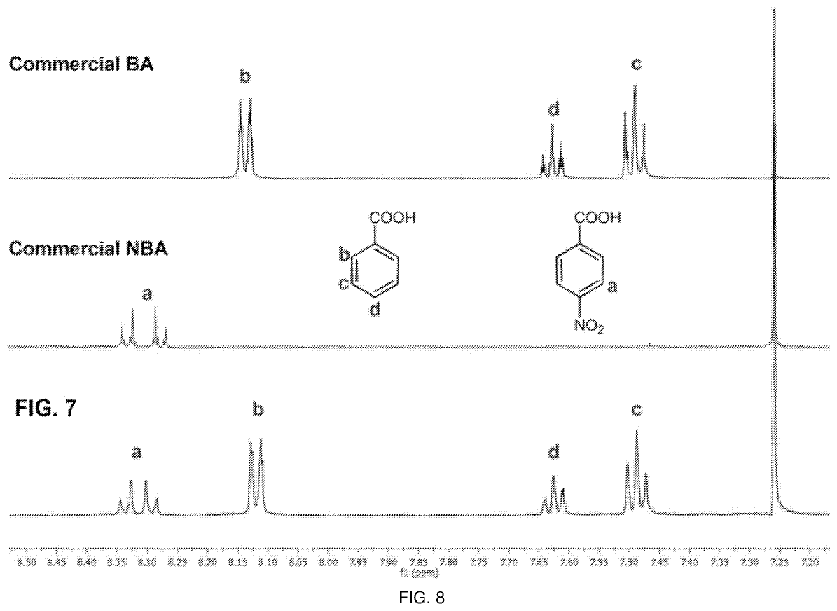

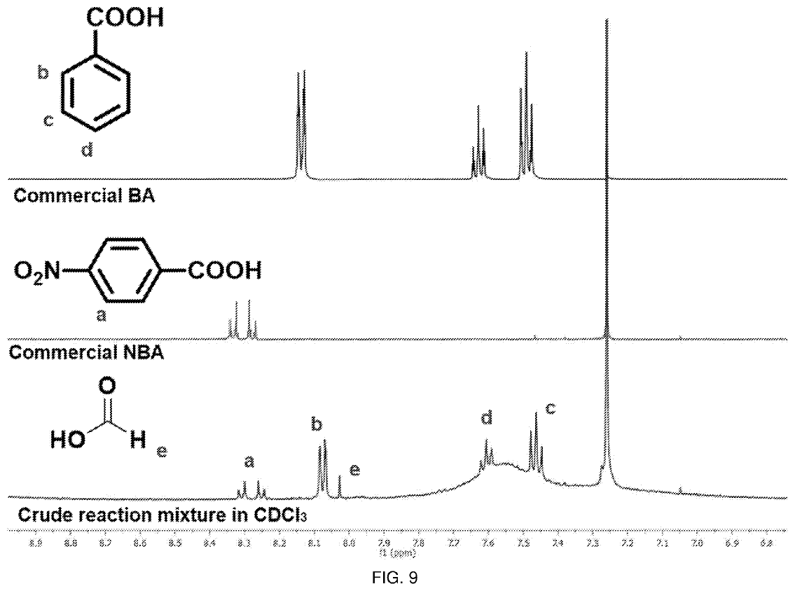

FIG. 7 shows ¹H NMR spectrum of pure BA/NBA products obtained after 1.04 g of styrofoam degradation in CDCl₃. FIG. 8 shows ¹H NMR spectrum of the pure BA/NBA products obtained from Example 4 (as shown in FIG. 7) stacked together with ¹H NMR spectrum of commercial BA and ¹H NMR spectrum of commercial NBA in CDCl₃. FIG. 9 shows ¹H NMR spectrum of a crude reaction mixture in CDCl₃, stacked together with ¹H NMR spectrum of commercial BA and ¹H NMR spectrum of commercial NBA in CDCl₃.

### Example 6: Yield Improvement

To optimize yield, further experiments were performed and it was found that the yields of BA and NBA can be improved by various optimization such as increasing reaction time (Table 3, Entries 1 and 3 compared with that from the 24 hour reaction in Table 1, Entry 1) or by adding the nitrate source over different intervals. Instead of a single addition of the HNO₃ nitrate source (Table 3, Entry 1), splitting the HNO₃ addition over two times, 24 hours apart, whilst keeping the total quantity of HNO₃ added the same (Table 3, Entry 2) resulted in a higher overall product yield.

TABLE 3
Extra conditions for yield optimization¹

| | | | | | | | | Yield | Yield |
|---|---|---|---|---|---|---|---|---|---|
| | | Nitrate | | Temperature | Time | Atmosphere | | of BA | of NBA |
| Entry | Catalyst | Source | Solvent | (° C.) | (hr) | (1 atm) | Extra Condition | (%) | (%) |
| 1 | THICA | HNO₃ | AcOH | 120 | 48 | Air | Single addition of HNO₃ | 41 | 11 |
| | (10 mol %) | (0.2 mL) | (2 mL) | | | | (0.2 mL at 0 hr) | | |
| 2 | THICA | HNO₃ | AcOH | 120 | 48 | Air | Separate addition of | 48 | 13 |
| | (10 mol %) | (0.2 mL) | (2 mL) | | | | HNO₃ (0.1 mL each for | | |
| | | | | | | | 0 hr and 24 hr intervals) | | |
| 3 | THICA | HNO₃ | AcOH | 120 | 72 | Air | Single addition of HNO₃ | 52 | 15 |
| | (10 mol %) | (0.2 mL) | (2 mL) | | | | (0.2 mL at 0 hr) | | |

¹Styrofoam was used as the PS source. Yield was determined by ¹H NMR using DCE as the internal standard.

### Example 7: Reaction Scheme

The method of converting a benzenoid-based polymer into oxidation/degradation products in accordance with various embodiments disclosed herein comprises one or more reaction steps. Examples of the reaction steps include, but are not limited to, hydrogen atom transfer, O₂ combination, NO₂ combination, homolytic cleavage, beta scission, chain continue, oxidation and the like. An example of the reaction steps involved in the conversion (or oxidative cleavage) of a benzenoid-based polymer is illustrated in Scheme 1. In this example, the benzenoid-based polymer used is polystyrene (PS).

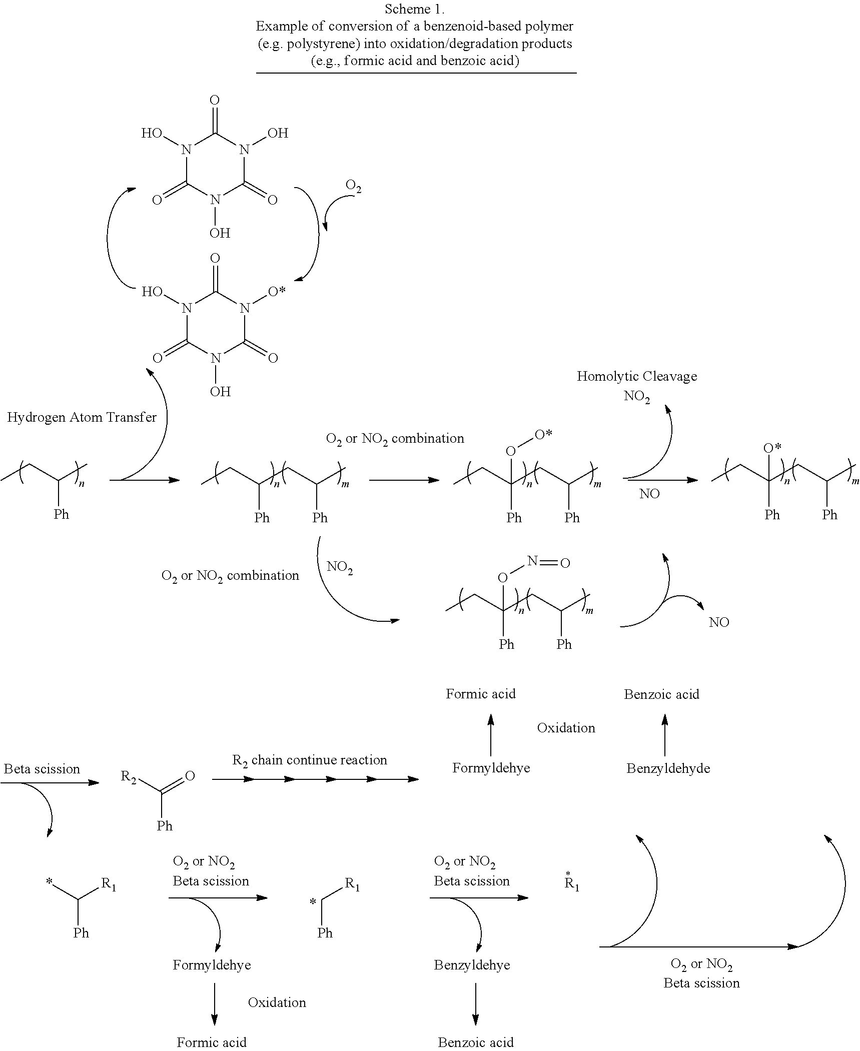

### SUMMARY

In summary, a method has been developed that allows polystyrene to be converted into useful chemicals such as BA and NBA via thermal oxidative degradation under mild conditions of 120° C. and 1 bar. The reaction proceeds well under a relatively short time of 24 hours with comparable yields to currently reported literatures. Embodiments of the method disclosed herein also have several advantages such as milder conditions, greener conditions, and resilience to different additives (particularly dyes) over currently known methods. Importantly, embodiments of the method disclosed herein are based on thermal catalysis, which is completely different from photo-catalysis. Accordingly, it is not obvious and there is no motivation for a person skilled in the art to arrive at embodiments of the presently disclosed method based on the prior art, which focus only on photocatalytic processes.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of converting a polymer comprising benzenoid rings into oxidation product(s), the method comprising:
oxidizing a benzenoid-based polymer source by adding a NOₓ source to the benzenoid-based polymer source to oxidize the polymer comprising benzenoid rings.

2. The method of claim 1, wherein the NOₓ source is selected from the group consisting of nitrous acid, nitrite salts, nitric acid, nitrate salts and combinations thereof.

3. The method of claim 2, wherein the NOₓ source is selected from the group consisting of nitric acid (HNO₃), sodium nitrate (NaNO₃), potassium nitrate (KNO₃), magnesium nitrate (Mg(NO₃)₂), calcium nitrate (Ca(NO₃)₂), barium nitrate (Ba(NO₃)₂), aluminium nitrate Al(NO₃)₃, copper(II) nitrate (Cu(NO₃)₂), zinc nitrate (Zn(NO₃)₂), tetra-n-butylammonium nitrate, cerium(III) nitrate (Ce(NO₃)₃), cerium(IV) ammonium nitrate (CAN) (NH₄)₂[Ce(NO₃)₆]), iron(III) nitrate (Fe(NO₃)₃), lanthanum nitrate (La(NO₃)₃) and combinations thereof.

4. The method of claim 1, wherein oxidizing is carried out in the presence of:
(i) an oxygen-containing gas;
(ii) optionally an environmentally benign solvent; and
(iii) optionally a catalyst.

5. The method of claim 1, wherein the polymer comprising benzenoid rings comprises:
(R1) one or more benzenoid repeating units represented by general formula (1A); and
(R2) optionally one or more non-benzenoid repeating units: 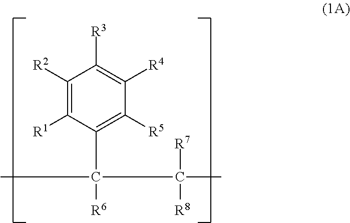
wherein
R¹ to R⁵ are each independently selected from the group consisting of H, optionally substituted alkyl, optionally substituted alkenyl, optionally substituted alkynyl, SO₃H, and halogen atoms;
R⁶ is H; and
R⁷ to R⁸ are each independently selected from the group consisting of H, optionally substituted alkyl, optionally substituted alkenyl and optionally substituted alkynyl.

6. The method of claim 1, wherein the oxidation product(s) comprises organic acid functional group.

7. The method of claim 6, wherein the oxidation product(s) comprises one or more compounds represented by general formula (2): 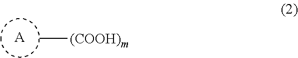
wherein
A is selected from the group consisting of H, optionally substituted alkyl, optionally substituted alkenyl, optionally substituted alkynyl, optionally substituted 5-membered ring and optionally substituted 6-membered ring; and
m=1 to 6.

8. The method of claim 7, wherein A is optionally substituted 6-membered ring and the oxidation product(s) comprises one or more compounds having general formula (2B): 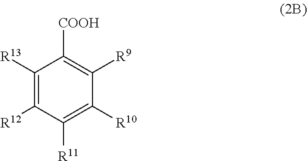
wherein
R⁹ to R¹³ are each independently selected from the group consisting of hydrogen (—H), nitro (—NO₂), carboxylic acid (—COOH), cyano (—CN), ester (—COOR), halogen (e.g., —F, —Cl, —Br, —I), hydroxy (—OH), alkoxy (—OR), sulfonic acid (—SO₃H), optionally substituted alkyl, optionally substituted alkenyl, and optionally substituted alkynyl, where R is an organic group.

9. The method of claim 7, wherein the oxidation product(s) comprises one or more of the following: terephthalic acid, phthalic acid, isophthalic acid, benzoic acid, nitrobenzoic acid and formic acid.

10. The method of claim 1, wherein the catalyst is present and comprises N-hydroxyl (—N—OH) groups.

11. The method of claim 10, wherein the catalyst comprises a structure selected from general formula (3), (4) and/or (5): 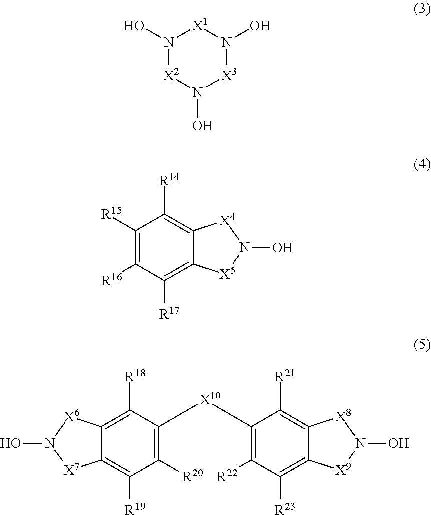
wherein
X¹ to X¹⁰ are each independently selected from the group consisting of CH₂, C(═O), S(═O)₂ and combinations thereof; and
R¹⁴ to R²³ are each independently selected from the group consisting of hydrogen (—H), nitro (—NO₂), carboxylic acid (—COOH), cyano (—CN), ester (—COOR), amine (—NR₂), halogen (e.g., —F, —Cl, —Br, —I), hydroxy (—OH), alkoxy (—OR), optionally substituted alkyl, optionally substituted alkenyl, optionally substituted alkynyl, and combinations thereof, where R is an organic group.

12. The method of claim 1, wherein the environmentally benign solvent is present and comprises a weak acid.

13. The method of claim 12, wherein the environmentally benign solvent comprises acetic acid.

14. The method of claim 1, wherein the oxygen-containing gas comprises an oxygen content that is from 20.5 vol % to 100 vol %.

15. The method of claim 1, wherein oxidizing is performed at a pressure that is from 0.5 bar to 10 bar.

16. (canceled)

17. The method of claim 1, wherein the oxidizing comprises adding from 0.10 mol % to 99.9 mol % of the benzenoid-based polymer source with respect to the reaction mixture.

18. (canceled)

19. The method of claim 1, wherein oxidizing is performed in the presence of from 0 mol % to 20 mol % of the catalyst with respect to the reaction mixture.

20. (canceled)

21. The method of claim 1, wherein the NOₓ source is mixed with the solvent in a molar ratio of from 1:1 to 1:20.

22. The method of claim 1, wherein the yield of the oxidation product(s) is modulated by adding the NOₓ source at different time points.

23. The method of claim 1, wherein the benzenoid-based polymer source comprises waste polystyrene.
